(19) United States
(12) Patent Application Publication
(10) Pub. No.: US 2021/0265006 A1
(43) Pub. Date: Aug. 26, 2021

(54) ARRAY BASED METHOD AND KIT FOR DETERMINING COPY NUMBER AND GENOTYPE IN PSEUDOGENES

(71) Applicant: AFFYMETRIX, INC., Carlsbad, CA (US)

(72) Inventors: Alan ROTER, Redwood City, CA (US); Jeanette SCHMIDT, Palo Alto, CA (US); Longyang WU, Emeryville, CA (US); Rama Rajaraja VARMA, Morgan Hill, CA (US); Zheng CAI, Santa Clara, CA (US); Thao-Nhi LE, San Jose, CA (US); Srividya MOHAN, San Mateo, CA (US); Kay AULL, Santa Clara, CA (US)

(21) Appl. No.: 17/262,311

(22) PCT Filed: Jul. 23, 2019
(86) PCT No.: PCT/US2019/043032

§ 371 Date: Jan. 22, 2021

Related U.S. Application Data
(60) Provisional application No. 62/702,827, filed on Jul. 24, 2018.
(60) Provisional application No. 62/875,363, filed on Jul. 17, 2019.

(51) Int. Cl. G16B 20/10; G16B 40/30

(52) U.S. Cl. CPC G16B 20/10; G16B 40/30

(57) ABSTRACT

Provided herein are methods and associated compositions, kits, systems, devices and instruments useful for genetic analysis where there is/are a sequence(s) similar to the gene of interest in a sample. In the methods, a combined copy number for related genes (e.g., a gene of interest and its pseudogene) can be determined via an assay. In addition, relative amounts of the related genes, i.e., a ratio of the related genes can be determined via the assay. Using the data of the combined copy number and the ratio of the related genes, the genotype of the gene of interest (as well as its pseudogene(s), if desired) can be determined with high accuracy.

### FIELD

The disclosure provides methods as all as associated compositions, kits, systems, devices and instruments useful for genetic analysis, including genotyping and copy number analysis of nucleic acids.

### BACKGROUND

Analysis of nucleic acid sequences, for example DNA and RNA samples obtained from a biological sample or organism has elicited significant interest in the research and health care communities. Using suitable methods, a collection of nucleic acid sequences can be analyzed to discern various genetic information, such as genotype and copy number variation, which can be important for diagnosis or screening of a disease or condition for the source of the nucleic acids as well as family members thereof. Analysis of certain nucleic acid sequences (e.g., clinically relevant genes or genes associated with pathogenic conditions or diseases) can be quite difficult if there are other nucleic acid sequences (e.g., pseudogenes) that are highly similar to the actually relevant genes. The challenge present in such an analysis (e.g., array-based or sequencing-based analysis) is partly because the signals detected from the analysis correspond to more than one gene. In some cases, it is often technically complex to assign the signals to their corresponding genes and statistically analyze the signals to determine the genetic information of individual genes separately.

Therefore, there is a need to develop improved methods (as all as associated compositions, kits, systems, devices and instruments) that leverages the genetic analysis to generate data with high accuracy, which can be used to both genotype and estimate copy number of a given locus or chromosome.

### SUMMARY

Described herein are methods and systems for analyzing a nucleic acid sample to detect differences in copy number of a target polynucleotide, such as a detection of copy number variants including deletion and insertion, as well as methods of genotyping such target polynucleotides, which are particularity useful when there are other sequences that have substantial sequence similarity to the target polynucleotides.

In one aspect, the disclosure provided herein relates to a method of genotyping nucleic acids of a sample. The method may include (a) providing the nucleic acids from a sample or amplified products thereof to an array, the array having a first set of probes and a second set of probes that hybridize to a first target polynucleotide and a second target polynucleotide, wherein the first set of probes hybridizes to a first region that has a sequence different in the first and second target polynucleotides and the second set of probes hybridize to a second region that is identical in the first and second target polynucleotides, and wherein the first and second target polynucleotides have sequence identity of at least 50%; (b) detecting a signal indicative of the hybridization of the first set of probes to the nucleic acids of the sample or amplified products thereof; (c) detecting a signal indicative of the hybridization of the second set of probes to the nucleic acids of the sample or amplified products thereof; and (d) determining the genotype of the nucleic acids of the sample by analyzing the signals.

In some embodiments, the first region has one or more base positions varying in the first and second target polynucleotides and a sequence that is identical in the first and second target polynucleotides and surrounding the varying position(s).

In some embodiments, the first set of probes hybridizes to a sequence that is immediately 5′ or 3′ of the varying position(s).

In some embodiments, the first set of probes terminates at a base immediately adjacent to the varying position(s).

In some embodiments, the first set of probes has a sequence that is complementary to the varying position(s).

In some embodiments, the first and second target polynucleotides are from different genes.

In some embodiments, the first and second target polynucleotides are not allelic variants of a gene.

In some embodiments, the analyzing step includes one or more of the following: (a) determining a combined copy number of the first and second target polynucleotides in the nucleic acids of the sample; and (b) determining a ratio of the amounts of the first and second target polynucleotides in the nucleic acids of the sample.

In some embodiments, the first and second target polynucleotides have sequence identity of at least about 55%, about 60%, about 65%, about 70%, about 75%, about 80%, about 85%, about 90%, about 95%, or about 99%.

In some embodiments, the nucleic acid of the sample has genomic DNA sequences obtained from the sample.

In some embodiments, the method further include amplifying the genomic DNA sequences obtained from the sample.

In some embodiments, the method further include amplifying the first and second target polynucleotides prior to the hybridization of the first and second probe sets to the nucleic acids of the sample.

In some embodiments, the method further includes fragmenting the nucleic acids or amplified products thereof.

In some embodiments, the fragmented nucleic acids or amplified products thereof are provided to the array.

In another aspect, the disclosure provided herein relates to a method of determining a carrier status of an individual for an autosomal recessive condition. The method may include (a) providing nucleic acids obtained from the individual or amplified products thereof to an array, the array having a first set of probes and a second set of probes that hybridize to a first target polynucleotide and a second target polynucleotide, wherein the first set of probes hybridizes to a first region that has a sequence different in the first and second target polynucleotides and the second set of probes hybridize to a second region that is identical in the first and second target polynucleotides, and wherein the first and second target polynucleotides have sequence identity of at least 50%; (b) detecting a signal indicative of the hybridization of the first set of probes to the nucleic acids of the individual or the amplified products thereof; (c) detecting a signal indicative of the hybridization of the second set of probes to the nucleic acids of the individual or the amplified products thereof; (d) genotyping the nucleic acids of the individual by analyzing the signals; and (e) determining the carrier status of the individual based on the genotype.

In some embodiments, the first region has one or more base positions varying in the first and second genes and a sequence surrounding the varying position(s).

In some embodiments, the first set of probes hybridizes to a sequence that is immediately 5′ or 3′ of the varying positions.

In some embodiments, the first set of probes terminates at a base immediately adjacent to the varying position(s).

In some embodiments, the first set of probes has a sequence that is complementary to the varying position(s).

In some embodiments, the first and second target polynucleotides are from different genes.

In some embodiments, the first and second target polynucleotides are not allelic variants of a gene.

In some embodiments, the analyzing step includes one or more of the following: (a) determining a combined copy number of the first and second target polynucleotides in the nucleic acids of the individual; and (b) determining a ratio of the amounts of the first and second target polynucleotides in the nucleic acids of the individual.

In some embodiments, the first and second target polynucleotides have sequence identity of at least about 55%, about 60%, about 65%, about 70%, about 75%, about 80%, about 85%, about 90%, about 95%, or about 99%.

In some embodiments, the nucleic acids obtained from the individual has genomic DNA.

In some embodiments, the method further includes amplifying the genomic DNA.

In some embodiments, the method further includes amplifying nucleic acids of the first and second target polynucleotides.

In some embodiments, the method further includes fragmenting the nucleic acids obtained from the individual or amplified products thereof, thereby generating fragmented nucleic acids.

In some embodiments, the method further includes providing the fragmented nucleic acids to the array.

In some embodiments, the method further includes determining the presence or absence of mutations, insertions, and/or deletions in the first target polynucleotide in the genome of the individual so as to determine the presence or absence of a functional copy of the first target polynucleotide in the individual.

In some embodiments, the method further includes determining the individual is a carrier for the autosomal recessive condition if the copy number of a functional first target polynucleotide from the individual is 1.

In another aspect, the disclosure provided herein relates to a kit for genotyping nucleic acids of a sample. The kit may contain an array having a first set of probes and a second set of probes that hybridize to a first target polynucleotide and a second target polynucleotide, wherein the first set of probes hybridizes to a first region that has a sequence different in the first and second target polynucleotides and the second set of probes hybridize to a second region that is identical in the first and second target polynucleotides, and wherein the first and second target polynucleotides have sequence identity of at least 50%.

In some embodiments, the first region contains one or more base positions varying in the first and second target polynucleotides and a sequence surrounding the varying position(s).

In some embodiments, the first set of probes hybridizes to a sequence that is immediately 5′ of the varying positions.

In some embodiments, the first set of probes terminates at a base immediately adjacent to the varying position(s).

In some embodiments, the first set of probes has a sequence that is complementary to the varying position(s).

In some embodiments, the first and second target polynucleotides are from different genes.

In some embodiments, the first and second target polynucleotides are not allelic variants of a gene.

In some embodiments, the first and second target polynucleotides have sequence identity of at least about 55%, about 60%, about 65%, about 70%, about 75%, about 80%, about 85%, about 90%, about 95%, or about 99%.

In some embodiments, the kit further contains instructions, in a computer-readable medium, having a code for receiving data indicative of hybridization of the first and second sets of probes to the nucleic acids of a sample or application products thereof, a code for determining a combined copy number of the first and second target polynucleotides in the nucleic acids of a sample, a code for determining a ratio of amounts of the first and second target polynucleotides from the nucleic acids of a sample, and a code for determining a genotype of the first and second target polynucleotides from the nucleic acids of a sample.

In still another aspect, the disclosure provided herein relates to a method of manufacturing an array for genotyping nucleic acids having first and second polynucleotides that have at least 50% of sequence identity. The method may include: (a) providing a first set of probes to a substrate, wherein the first set of probes hybridizes to a first region that has a sequence different in the first and second polynucleotides; and (b) providing a second set of nucleic acid sequences to the substrate, wherein the second set of probes hybridize to a second region that is identical in the first and second polynucleotides.

In some embodiments, the first and second sets of probes are synthesized on a substrate or attached to the substrate after being synthesized.

In some embodiments, the first region contains one or more base positions varying in the first and second polynucleotides and a sequence surrounding the varying position(s).

In some embodiments, the first set of probes hybridizes to a sequence that is immediately 5′ of the varying positions.

In some embodiments, the first set of probes terminates at a base immediately adjacent to the varying position(s).

In some embodiments, the first set of probes contains a sequence that is complementary to the varying position(s).

In some embodiments, the first and second polynucleotides are from different genes.

In some embodiments, the first and second polynucleotides are not allelic variants of a gene.

In some embodiments, the first and second polynucleotides have sequence identity of at least about 55%, about 60%, about 65%, about 70%, about 75%, about 80%, about 85%, about 90%, about 95%, or about 99%.

In still another aspect, the disclosure provided herein relates to a computer-implemented method for genotyping a mixture of nucleic acids, the mixture having a first target polynucleotide and a second target polynucleotide having a sequence identity of at least 50% to the first target polynucleotide. The method may include: obtaining, by a computer having a processor, first data of an intensity measurement from a first set of probes, wherein the first set of probes targets a sequence that is different in the first and second target polynucleotide sequences; obtaining, by the computer, second data of an intensity measurement from a second set of probes, wherein the second set of probes targets a sequence that is identical in the first and second target polynucleotides sequences; determining, by the processor, from the first data a ratio of the first and second target polynucleotides in the mixture; determining, by the processor, from the second data a combined copy number of the first and second target polynucleotides in the mixture; and determining, by the processor, a genotype of at least one of the first and second target polynucleotides.

In some embodiments, the first and second sets of probes are provided in an array.

In some embodiments, the first and second sets of probes hybridize to the target polynucleotides on the array.

In some embodiments, the ratio of the first and second target polynucleotides is a ratio of the first and second target polynucleotides in a human genome.

In some embodiments, the combined copy number of the first and second target polynucleotides is a combined genomic copy number of the first and second target polynucleotides in a human genome.

In some embodiments, the first and second target polynucleotides are from different genes.

In some embodiments, the first and second target polynucleotides are not allelic variants of a gene.

In some embodiments, the target polynucleotides are Survival Motor Neuron 1 (SMN1) and Survival Motor Neuron 2 (SMN2) genes or part thereof.

In some embodiments, the first target polynucleotide is found in the SMN2 gene and a variant of SMN1 gene with a mutation in and around exon 7.

In some embodiments, the second target polynucleotide is found in the SMN1 gene.

In some embodiments, the first set of probes has at least four probe sets and each probe set corresponds to a sequence that is different in SMN1 and SMN2 genes.

In some embodiments, the at least four probe sets targeting variants in and around exon 7 of the SMN1 gene target the following regions: a region containing chromosome 5:70,247,773C>T site, a region containing chromosome 5: 70,247,921A>G site, a region containing chromosome 5: 70,248,036A>G site, and a region containing chromosome 5: 70,248,501G>A.

In some embodiments, the nucleotide sequences are human sequences.

In some embodiments, the method further includes receiving data of signals from the array, wherein in the first set of probes the first target polynucleotide is reported; calculating an average intensity value for the probe sets and determining a standard deviation between the average intensity values; calculating a raw frequency of the target polynucleotides; calculating a centered frequency of the target polynucleotides from the respective raw frequency; calculating a scaled and centered frequency of the target polynucleotides from the respective centered frequency; calculating a median frequency of the target polynucleotides from an affinity value of each probe set for the target polynucleotides and predicted copy number (CN); delineating hyperplanes corresponding to presence of no copy of the target polynucleotides in the mixture, one copy of the target polynucleotides gene in the mixture, and two copies of the target polynucleotides in the mixture; and correlating quantity of probe set clusters within the hyperplanes as a statistical indication of the number of copies of the target polynucleotides in the mixture.

In some embodiments, the method further includes: scaling the scaled and centered frequency by: setting the scaled and centered frequency to 1 in response to the scaled and centered frequency being greater than 1; and setting the scaled and centered frequency to 0 in response to the scaled and centered frequency being less than 0; and determining the direction of the frequency by subtracting a median frequency for the first target polynucleotide, and using the median frequency value for the second target polynucleotide.

In some embodiments, calculating the raw frequency for the probe sets further includes dividing an intensity for the second target polynucleotide by the sum of an intensity for the first target polynucleotide and an intensity for the second target polynucleotide.

In some embodiments, calculating the raw frequency for the probe sets further includes dividing an intensity for the first target polynucleotide by the sum of an intensity for the first target polynucleotide and an intensity for the second target polynucleotide.

In some embodiments, calculating the centered frequency for the probe sets from the raw frequency further includes subtracting the standard deviation from the raw frequency and then adding an ideal frequency ratio of 0.5, the ideal frequency being the frequency between the first and second target polynucleotides.

In some embodiments, calculating the scaled and centered frequency for the probe sets from the centered frequency further includes: multiplying the difference between the centered frequency and a first alpha cutoff value by a first scaling factor and then subtracting this value from the first alpha cutoff value, in response to the centered frequency being less than the first alpha cutoff value; multiplying the difference between the centered frequency and a second alpha cutoff value by a second scaling factor and then adding this value to the second alpha cutoff value, in response to the centered frequency being greater than the second alpha cutoff value; and identifying the centered frequency as the scaled and centered frequency in response to the centered frequency being equal or within a range formed by the first alpha cutoff value and the second alpha cutoff value.

In some embodiments, the method further includes: plotting the scaled and centered frequency for the probe sets against their predicted copy number on a graph; delineating the hyperplanes in the graph corresponding to presence of no copy of the target polynucleotides in the mixture, one copy of the target nucleoids in the mixture, and two copies of the target nucleotides in the mixture; and correlating the quantity of probe set clusters within the hyperplanes as the statistical indication of the number of copies of the target nucleotides in the mixture.

In some embodiments, the method further includes normalizing the raw frequency for each of the probe sets.

In some embodiments, normalizing the raw frequency for the probe sets further includes: calculating a centered frequency for the probe sets from the raw frequency by subtracting the standard deviation from the raw frequency and then adding an ideal frequency ratio of 0.5, the ideal frequency being the raw frequency between the first and second target polynucleotides; calculating a scaled and centered frequency for the probe sets from the centered frequency by: multiplying the difference between the centered frequency and a first alpha cutoff value by a first scaling factor and then subtracting this value from the first alpha cutoff value, in response to the centered frequency being less than the first alpha cutoff value; multiplying the difference between the centered frequency and a second alpha cutoff value by a second scaling factor and then adding this value to the second alpha cutoff value, in response to the centered frequency being greater than the second alpha cutoff value; and identifying the centered frequency as the scaled and centered frequency in response to the centered frequency being equal or within a range formed by the first alpha cutoff value and the second alpha cutoff value.

In still another aspect, the disclosure provided herein relates to a method including: receiving a probe set data for an array having a first set of probes and a second set of probes, the first set of probes targeting a variable sequence of first and second target polynucleotides and the second set of probes targeting an identical sequence of the target polynucleotides, the data having an average signal intensity for the target polynucleotide to each probe sets, a standard deviation of the average signal intensity for each probe set, a first scaling factor, a second scaling factor, and a copy number region; calculating a raw frequency of the target polynucleotides from the average signal intensity from the probe sets; calculating a centered frequency of the target polynucleotides from the respective raw frequency, an ideal frequency ratio, and the standard deviation; calculating a scaled and centered frequency of the target polynucleotides from the respective centered frequency, a first alpha cutoff value, a second alpha cutoff value, the first scaling factor, and the second scaling factor; calculating a median frequency of the target polynucleotides from an affinity value of each probe set for the target polynucleotides and predicted copy number (CN); delineating hyperplanes corresponding to presence of no copy of the target polynucleotides, one copy of the target polynucleotides, and two copies of the target polynucleotides; and correlating quantity of probe set clusters within the hyperplanes as a statistical indication of the number of copies of the target polynucleotides.

In some embodiments, the copy number of the target polynucleotides is a genomic copy number of the target polynucleotides in a human genome.

In some embodiments, the first and second target polynucleotides have sequence identity of at least 50%.

In some embodiments, the first and second target polynucleotides are from different genes.

In some embodiments, the first and second target polynucleotides are not allelic variants of a gene.

In some embodiments, the target polynucleotides are Survival Motor Neuron 1 (SMN1) and Survival Motor Neuron 2 (SMN2) genes or part thereof.

In some embodiments, the first target polynucleotide is found in the SMN2 gene and a variant of SMN1 gene with a mutation in and around exon 7.

In some embodiments, the second target polynucleotide is found in the SMN1 gene.

In some embodiments, the first set of probes has at least four probe sets and each probe set corresponds to a sequence that is different in SMN1 and SMN2 genes.

In some embodiments, the at least four probe sets targeting variants in and around exon 7 of the SMN1 gene target the following regions: a region containing chromosome 5:70,247,773C>T site, a region containing chromosome 5: 70,247,921A>G site, a region containing chromosome 5: 70,248,036A>G site, and a region containing chromosome 5: 70,248,501G>A.

In some embodiments, the method further includes: scaling the scaled and centered frequency by: setting the scaled and centered frequency to 1 in response to the scaled and centered frequency being greater than 1; and setting the scaled and centered frequency to 0 in response to the scaled and centered frequency being less than 0; and determining the direction of the raw frequency by subtracting a median frequency value for the first target polynucleotide and using the median frequency value for the second target nucleotide.

In some embodiments, calculating the raw frequency for the probe sets further includes dividing an intensity for the second target polynucleotide, by the sum of an intensity for the first target polynucleotide and the intensity for the second target polynucleotide.

In some embodiments, calculating the raw frequency for the probe sets further includes dividing an intensity for the first target polynucleotide by the sum of the intensity for the first target polynucleotide and an intensity for the second target polynucleotide.

In some embodiments, calculating the centered frequency for the probe sets from the raw frequency further includes subtracting the standard deviation from the raw frequency and then adding the ideal frequency ratio of 0.5, the ideal frequency being the raw frequency between the first and second target polynucleotides.

In some embodiments, calculating the scaled and centered frequency for the probe sets from the centered frequency further includes: multiplying the difference between the centered frequency and the first alpha cutoff value by the first scaling factor and then subtracting this value from the first alpha cutoff value, in response to the centered frequency being less than the first alpha cutoff value; multiplying the difference between the centered frequency and the second alpha cutoff value by the second scaling factor and then adding this value to the second alpha cutoff value, in response to the centered frequency being greater than the second alpha cutoff value; and identifying the centered frequency as the scaled and centered frequency, in response to the centered frequency being equal or within a range formed by the first alpha cutoff value and the second alpha cutoff value.

In some embodiments, the method further includes: plotting the scaled and centered frequency for the target polynucleotides against their predicted copy number on a graph; delineating the hyperplanes in the graph corresponding to presence of no copy of the target polynucleotides, one copy of the target polynucleotides, and two copies of the target polynucleotides; and correlating the quantity of probe set clusters within the hyperplanes as the statistical indication of the number of copies of the target polynucleotides in a human genome.

In some embodiments, the target polynucleotides are human sequences.

In still another aspect, the disclosure provided herein relates to a method of identifying a carrier genotype of an autosomal recessive condition in a subject. The method may include: obtaining first data for a first set of probes targeting a first marker sequence that is different in a first polynucleotide sequence and a second polynucleotide sequence, wherein the first and second polynucleotide sequences have sequence identity of at least 50% and the autosomal recessive condition is caused at the absence of functional copies of the first polynucleotide sequence in a genome; obtaining second data for a second set probes targeting a second marker sequence that is identical in the first polynucleotide sequence and the second polynucleotide sequence; calculating a copy number for at least one polynucleotide sequences and a ratio identifying relative presence of the first and second polynucleotide sequences from the first data and the second data; identifying a carrier genotype when the copy number of the first polynucleotide sequence is less than 2; and/or when the ratio indicates a higher presence of the second polynucleotide sequence relative to the first polynucleotide sequence.

INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic diagram showing autosomal recessive inheritance.

FIG. 2 illustrates a spinal muscular atrophy (SMA) phenotype manifestation 100 in accordance with one embodiment.

FIG. 3 illustrates a Survival Motor Neuron 1 (SMN1) genotypes 200 in accordance with one embodiment.

FIG. 4 illustrates a genome browser 300 in accordance with one embodiment.

FIG. 5 illustrates a genomic browser 400 in accordance with one embodiment.

FIG. 6 illustrates an SMN1 base sequence 500 in accordance with one embodiment.

FIG. 7 illustrates a sequence alignment in accordance with one embodiment.

FIG. 8 illustrates Survival Motor Neuron 1 (SMN1) and Survival Motor Neuron 2 (SMN2) sequence variant genotypes 700 in accordance with one embodiment.

FIG. 9 illustrates a copy number determination process 800 in accordance with one embodiment.

FIG. 10 illustrates a system 900 in accordance with one embodiment.

FIG. 11 illustrates a plot 1400 in accordance with one embodiment.

FIG. 12 illustrates a plot 1500 in accordance with one embodiment.

FIG. 13 is an example block diagram of a computing device 1600 that may incorporate embodiments of the present disclosure.

FIG. 14 shows the distribution of copy number of SMN1 and SMN2 for 96 representative samples.

FIG. 15 shows the results identifying carriers for SMA.

FIG. 16 shows an example of copy number display for both SMN1 and SMN2. The data shown at y-axis value of 1.5 or below may indicate the samples suspected to be SMA carrier in this illustrated example.

### DETAILED DESCRIPTION

The present disclosure has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

Throughout this disclosure, various aspects of this disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present disclosure may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

DEFINITIONS

As used in this application, the singular form “a,” “an,” and “the” include plural references unless the context clearly dictates otherwise. For example, the term “an agent” includes a plurality of agents, including mixtures thereof.

All references cited herein are incorporated herein in their entireties for all their purposes. To the extent any reference includes a definition or uses a claim term in a manner inconsistent with the definitions and disclosure set forth herein, the definitions and disclosure of this application will control.

As may be used herein, the terms, in a singular or plural form, “nucleic acid(s),” “nucleic acid molecule(s),” “nucleic acid oligomer(s),” “oligonucleotide(s),” “nucleic acid sequence(s),” “nucleic acid fragment(s)” and “polynucleotide(s)” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

“Percentage of sequence identity” or “percentage of sequence similarity” is determined by comparing two optimally aligned sequences over a comparison window, wherein the portion of the polynucleotide or polypeptide sequence in the comparison window may comprise mutations, additions or deletions (i.e., gaps) as compared to the reference sequence (which does not comprise mutations, additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” and “similar” or percent “similarity,” in the context of two or more nucleic acids sequences, refer to two or more sequences or subsequences that are the same or have a specified percentage of nucleotides that are the same (i.e., about 50% identity, preferably 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have mutations and/or substitutions. In some embodiments, the preferred algorithms can account for gaps and the like.

The terms “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

As used here, “gene” refers to a sequence of DNA or RNA that codes for a molecule that has a function. Therefore, a sequence of DNA or RNA that is translated into a polypeptide forms a gene. In addition, any regulatory sequences, e.g., promoter, enhancer, 5′ and 3′ regulatory sequences of DNA, intron and many others that has any function in cells (including, but is not limited to, functions in DNA replication, transcription and translation) are considered part of a gene. Also, there are genes such as miRNA and siRNA that are not translated and provides certain functions in cells are also considered genes.

As used herein, “allele” refers to one specific form of a nucleic acid sequence (such as a gene) within a cell, an individual or within a population, the specific form differing from other forms of the same gene in the nucleic acid sequence of at least one, and frequently more than one, variant sites within the sequence of the gene. The sequences at these variant sites that differ between different alleles are termed “variances,” “polymorphisms,” or “mutations.” The variants in the sequence can occur as a result of SNPs, combinations of SNPs, haplotype methylation patterns, insertions, deletions, and the like. An allele may comprise the variant form of a single nucleotide, a variant form of a contiguous sequence of nucleotides from a region of interest on a chromosome, or a variant form of multiple single nucleotides (not necessarily all contiguous) from a chromosomal region of interest. At each autosomal specific chromosomal location or “locus” an individual possesses two alleles, one inherited from one parent and one from the other parent, for example one from the mother and one from the father. An individual is “heterozygous” at a locus if it has two different alleles at that locus. An individual is “homozygous” at a locus if it has two identical alleles at that locus.

As used herein, “genome” designates or denotes the complete, single-copy set of genetic instructions for an organism as coded into the DNA of the organism. A genome may be multi-chromosomal such that the DNA is cellularly distributed among a plurality of individual chromosomes. For example, in humans there are 22 pairs of chromosomes plus a gender associated XX or XY pair.

As used herein, “polymorphism” refers to the occurrence of two or more genetically determined alternative sequences in a population. The alternative sequences can include alleles (e.g., naturally occurring variants) or spontaneously arising mutations that only occur in one or few individual organisms. A “polymorphic site” can refer to the nucleic acid position(s) at which a difference in nucleic acid sequence occurs. A polymorphism may comprise one or more base changes, an insertion, a repeat, or a deletion. A polymorphic locus may be as small as one base pair. Polymorphic sites include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements. The first identified variant or allelic form is arbitrarily designated as the reference form and other variant or allelic forms are designated as alternative or variant or mutant alleles. The variant or allelic form occurring most frequently in a selected nucleic acid population is sometimes referred to as the wildtype form. When a gene encoding a polypeptide is concerned, the wild type may refer to the sequence of the gene that is most frequent and encodes a polypeptide exhibiting the expected activity. Diploid organisms may be homozygous or heterozygous for allelic forms. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens. SNPs are positions at which two alternative bases occur at appreciable frequency (>1%) in the human population, and are the most common type of human genetic variation.

As used herein, “an array” or “a microarray” comprises a support with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251:767-777 (1991). Each of which is incorporated by reference in its entirety for all purposes. The probes can be of any size or sequence, and can include synthetic nucleic acids, as well as analogs or derivatives or modifications thereof, as long as the resulting array is capable of hybridizing under any suitable conditions with a nucleic acid sample with sufficient specificity as to discriminate between different target nucleic acid sequences of the sample. In some embodiments, the probes of the array are at least 5, 10, 20, 30, 40, 50, 60, 70 or 80 nucleotides long. In some embodiments, the probes are no longer than 25, 30, 50, 75, 100, 150, 200 or 500 nucleotides long. For example, the probes can be between 10 and 100 nucleotides in length.

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on three-dimensional matrices, beads, gels, polymeric surfaces, fibers such as optical fibers, glass or any other appropriate substrate. (See U.S. Pat. Nos. 5,770,358, 5,789,162, 5,708,153, 6,040,193 and 5,800,992, which are hereby incorporated by reference in their entirety for all purposes.)

In some embodiments, arrays useful in connection with the methods and systems described herein include commercially available from Thermo Fisher Scientific (formerly Affymetrix) under the brand name GeneChip® and are directed to a variety of purposes, including genotyping and gene expression monitoring for a variety of eukaryotic and prokaryotic species. Methods for preparing a sample for hybridization to an array and conditions for hybridization are disclosed in the manuals provided with the arrays, for example, those provided by the manufacturer in connection with products, such as the OncoScan® FFPE Assay Kit, and related products.

As used herein, “genotyping” refers to the determination of the nucleic acid sequence information from a nucleic acid sample at one or more nucleotide positions. The nucleic acid sample may contain or be derived from any suitable source, including the genome or the transcriptome. In some embodiments, genotyping may comprise the determination of which allele or alleles an individual carries at one or more polymorphic sites. For example, genotyping may include or the determination of which allele or alleles an individual carries for one or more SNPs within a set of polymorphic sites. For example, a particular nucleotide in a genome may be an A in some individuals and a B in other individuals. Those individuals who have an A at the position have the A allele and those who have a B have the B allele. In a diploid organism the individual will have two copies of the sequence containing the polymorphic position so the individual may have an A allele and a B allele or alternatively two copies of the A allele or two copies of the B allele. Those individuals who have two copies of the A allele are homozygous for the A allele, those individuals who have two copies of the B allele are homozygous for the B allele, and those individuals who have one copy of each allele are heterozygous. Thus, in some embodiments, genotyping includes determination of allelic composition (e.g., AA, BB or AB) of a gene in a nucleic acid sample or of an individual. In some embodiments, genotyping includes determination of allelic composition of a plurality of genes (i.e., two or more genes). Therefore, in an example where two genes (e.g., first and second genes) are interrogated, and the first gene can have A and/or B alleles and the second gene can have C and/or D alleles, the methods herein can determine the genotypes of both genes, e.g., AACC, AADD, BBCC, or BBDD (if both genes are homozygous) or AACD, BBCD, ABCC, ABDD, or ABCD (if at least one gene is heterozygous). In some embodiments, genotyping includes detecting a single nucleotide mutation that arises spontaneously in the genome, amongst a background of wild-type nucleic acid. In some embodiments, one or more polynucleotides (or a portion or portions of the polynucleotide, its amplification products, or complements thereof) that contain a sequence of interest (e.g., one or more SNP or mutation) can be processed by other techniques such as sequencing. Therefore, in some embodiments, the polynucleotides can be sequenced for genotyping or determining the presence or absence of the polymorphism or mutation. The sequencing can be done via various methods available in the art, e.g., Sanger sequencing method that can be performed by, e.g., SeqStudio® Genetic Analyzer from Applied Biosystems) or Next Generation Sequencing (NGS) method, e.g., Ion Torrent NGS from Thermo Fisher or Illumina NGS.

As used herein, “chromosomal abnormalities” or “chromosomal abnormality” can include any genetic abnormality including mutations, insertions, additions, deletions, translocation, point mutation, trinucleotide repeat disorders and/or SNPs. While the present disclosure describes certain examples and embodiments related to the detection of chromosomal abnormalities in a carrier who is not be substantially affected by the abnormalities, it will be appreciated that the methods and system described herein can be used to detect chromosomal abnormalities in a patient who is affected by or has a high risk of the abnormalities.

As used herein, “sample” that is obtained from a biological sample or an organism includes, but is not limited to, any number of tissues or fluids, such as blood, urine, serum, plasma, lymph, saliva, stool, and vaginal secretions, of virtually any organism. In some embodiments, a sample obtained from an organism can be a mammalian sample. And in some embodiments, a sample obtained from an organism can be a human sample.

The term “mPCR” herein may refer to multiplex PCR, a molecular biology technique for amplification of multiple targets in a single PCR experiment. In a multiplexing assay, more than one target sequence can be amplified by using multiple primer pairs in a reaction mixture.

The term “CarrierScan” herein may refer to a genotyping product available from Thermofisher Corp. CarrierScan includes CarrierScan Assay that amplifies a precise target DNA of interest and the CarrierScan Array, an allele-specific oligonucleotide array provides a single color readout.

The term “annealing” herein may refer to pairing complementary sequences of single-stranded DNA or RNA with hydrogen bonds to form a double-stranded polynucleotide.

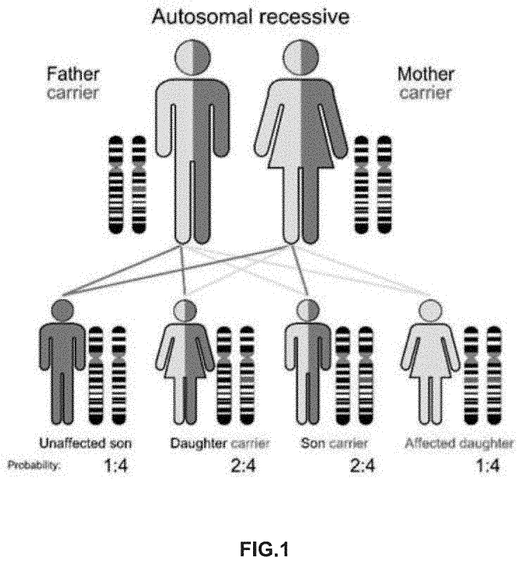

The term “carrier” herein may refer to a genotype associated with a homozygous recessive trait that is not currently expressed due to the presence of at least one functioning allele. When an individual carrying the homozygous recessive trait is crossed with another carrier 50% of the progeny will express the trait. See FIG.1.

The term “exon” herein may refer to part of a gene that will encode a part of the final mature RNA produced by that gene after introns have been removed by RNA splicing. The term exon refers to both the DNA sequence within a gene and to the corresponding sequence in RNA transcripts. In RNA splicing, introns are removed and exons are covalently joined to one another as part of generating the mature messenger RNA. Just as the entire set of genes for a species constitutes the genome, the entire set of exons constitutes the exome.

The term “DNase” herein may refer to deoxyribonuclease, an enzyme that catalyzes the hydrolytic cleavage of phosphodiester linkages in the DNA backbone, thus degrading DNA. Deoxyribonucleases are one type of nuclease, a generic term for enzymes capable of hydrolyzing phosphodiester bonds that link nucleotides. A wide variety of deoxyribonucleases are known, which differ in their substrate specificities, chemical mechanisms, and biological functions.

The term “duplication event” herein may refer to a mechanism through which new genetic material is generated during molecular evolution. It can be defined as any duplication of a region of DNA that contains a gene. Gene duplications can arise as products of several types of errors in DNA replication and repair machinery as well as through fortuitous capture by selfish genetic elements. Common sources of gene duplications include ectopic recombination, retro-transposition event, aneuploidy, polyploidy, and replication slippage

The term “circuitry” herein may refer to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

The term “firmware” herein may refer to software logic embodied as processor-executable instructions stored in read-only memories or media.

The term “hardware” herein may refer to logic embodied as analog or digital circuitry.

The term “logic” herein may refer to machine memory circuits, non-transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

The term “software” herein may refer to logic implemented as processor-executable instructions in a machine memory (e.g. read/write volatile or nonvolatile memory or media).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

### Genetic Analysis

Genetic analysis is of paramount importance in a number of healthcare and medical applications. Genetic analysis can provide information on one or more genes that are associated with a disease or condition of interest. For example, the genetic analysis can provide genotype of the clinically relevant gene(s) (or the gene(s) of interest) as well as presence or absence of any genetic abnormalities such as copy number variation, deletion, insertion, duplication, and mutation in chromosomes. The genetic analysis can be very difficult when there are other sequences that are highly similar to the gene(s) of interest. In some cases, there are pseudogenes, which are segments of DNA related to the genes of interest. In many cases, pseudogenes have lost at least some functionality, relative to the actual (or real) gene, in cellular gene expression or protein-coding ability. Pseudogenes often result from the accumulation of multiple mutations within a gene whose product is not required for the survival of the organism, but can also be caused by genomic copy number variation (CNV) where segments are duplicated or deleted. Although not fully functional, pseudogenes can be functional, similar to other kinds of noncoding DNA, which can perform regulatory functions. Given the substantial sequence similarity between the pseudogenes and actual genes (e.g., the clinically relevant genes or genes associated with genetic disease or condition), both sequences generate signals in an assay such as array and sequencing, and processing such mixed signals is technically challenging when compared to a case where only the actual genes are present in the genome. Methods, compositions, systems, devices and instruments provided herein are particularity useful in genetic analysis where there are a plurality of related genes in the genome.

In some embodiments, the present disclosure provides methods of genetic analysis. In some embodiments, the methods are useful to genotyping nucleic acids that has two or more related sequences (e.g., the sequences having substantial sequence similarity). For example, the method can be used to genotype a target gene that has one or more pseudogene sequence in the genome. Genotyping as well as copy number determination in such a situation can be technically challenging. Assays for genotyping and copy number determination, e.g. array-based, sequencing-based or PCR-based methods, often rely on interrogation of a region that is uniquely present in a target sequence. These assays typically interrogate a number of regions of the target sequence in order to provide a statistically meaningful and accurate result. Taking a genotyping assay as an example, a number of polymorphic sites that are different in alleles for the target gene can be interrogated via an array-, sequencing- or PCR-based approach and statistical analysis of multiple data points generated from the various polymorphic sites can provide a comprehensive and reliable genotype of the target gene. Also in an example of copy number determination, a number of regions that are unique to the target sequence can be interrogated and the numerous data points can be compared to that of a reference chromosome. In these assays, one or a few data points may not be sufficient to provide a reliable result as a variation for each data point is relatively large. Measuring a sufficient number of data points (e.g., 5 or more) and determining a prevailing relation of the multiple data points can provide a reliable result of genotyping and copy number for the target gene. As such, making sure that each data point represents a single gene of interest is important to have a successful and reliable genotyping and copy number determination in the foregoing types of assays. However, if there are more than one sequences that are highly similar to each other, e.g. a gene and its pseudogene exist in a genome, interpretation of the data and processing the same to genotype individual genes can be highly technically challenging. This is because each data point may be generated from a mixture of two genes and it is not possible to statistically analyze these mixed-up data and provide the result for individual genes separately. Therefore, with this complexity of the sequences in a sample, it is often impossible to determine a genotype or copy number for a target gene using the assays available in the field. To overcome the above challenges and provide a reliable genetic analysis result including genotyping and copy number for a gene of interest, provided herein are methods and associated compositions, kits, systems, devices and instruments useful for genetic analysis, especially where there is/are a sequence(s) similar to the gene of interest in a sample. In some embodiments, a copy number for related genes (e.g., a gene of interest and its pseudogene(s)), i.e., a “combined” copy number for related genes is determined via an assay. In addition, relative amounts of related genes, i.e., a ratio of related genes is determined via the assay. Using the data of the combined copy number and the ratio of the related genes, the genotype of the gene of interest (as well as its pseudogene(s), if desired) can be determined with high accuracy.

In some embodiments, provided herein are the methods of genotyping a plurality of polynucleotides (e.g., first and second polynucleotides), which has the following steps: (a) providing the nucleic acids from a sample or amplified products thereof to an array, which has a first set of probes and a second set of probes that hybridize to a first target polynucleotide and a second target polynucleotide, (b) detecting a signal indicative of the hybridization of the first set of probes to the nucleic acids of the sample or amplified products thereof, (c) detecting a signal indicative of the hybridization of the second set of probes to the nucleic acids of the sample or amplified products thereof and (d) determining the genotype of the nucleic acids of the sample by analyzing the signals. In some embodiments, the first set of probes hybridizes to a first region that has a sequence different in the first and second target polynucleotides. In some embodiments, the second set of probes hybridizes to a second region that is identical in the first and second target polynucleotides. The first and second target polynucleotides may have sequence identity of at least 50%.

In some embodiments, the methods according to the present disclosure is used to genotype nucleic acids that has at least two target polynucleotides, e.g., first and second polynucleotides that have sequence similarity. In some embodiments, the first and second polynucleotides have sequence similarity of at least about 50%, about 55%, about 60%, about 65%, about 70%, about 75%, about 80%, about 85%, about 90%, about 95%, about 99%, about 99.99% or any intervening percentage of the foregoing. In some embodiments, the first and second polynucleotides are not allelic variants of a single gene. In some embodiments, the first and second polynucleotides are two separate genes. In some embodiments, the first polynucleotide is a gene that has autosomal recessive inheritance, causing a genetic condition or disease upon loss of both active copies. In some of such embodiments, the second polynucleotide is a gene, e.g. a pseudogene that is similar in sequence to the first polynucleotide (or the first gene) but has no or less activity than the first gene.

In some embodiments, the two or more target polynucleotides that can be genotyped by the methods of the present disclosure have a region that is common (or identical) in the target polynucleotides and another region that is different (or varying) in the target polynucleotides. In some embodiments, the common region and different region are independently about 10 bases to about several hundred bases. In some embodiments, the common and different regions are independently about 10 bases, about 20 bases, about 30 bases, about 40 bases, about 50 bases, about 60 bases, about 70 bases, about 80 bases, about 90 bases, about 100 bases, about 110 bases, about 120 bases, about 130 bases, about 140 bases, about 150 bases, about 160 bases, about 170 bases, about 180 bases, about 190 bases, about 200 bases, about 250 bases, about 300 bases, about 400 bases, about 500 bases or any intervening number of bases of the foregoing. In some embodiments, all the bases in the common region are identical in the target polynucleotides. In some embodiments, in the varying region some of the bases are different in the target polynucleotides while some other bases are identical. In other words, the varying region has at least one or more bases that are different in the target polynucleotides and also a sequence near (or surrounding) the variable bases that are identical in the target polynucleotides. In some embodiments where two related genes are genotyped, variable bases in the varying region contain one or more bases mutated, deleted, or inserted in one of the gene but not the other gene. In some embodiments, the variable bases can be found anywhere in the genome that constitutes the gene, including not only a coding region(s) but also non-coding region(s) (e.g., 5′ and 3′ regulatory regions including promoter, enhancer and 5′ and 3′ untranslated region (UTR)) and introns. In some embodiments, the target polynucleotides include non-coding sequences such as microRNA (miRNA) and small interfering RNA (siRNA). Therefore, the method for genotyping provided herein is not limited to coding sequences but includes interrogation of non-coding sequences that are present anywhere in the genomes.

In some embodiments, the methods of the present disclosure for genotyping a plurality of target polynucleotides (e.g., first and second polynucleotides) utilize an array that has a number of probes. In some embodiments, the array has a first set of probes and a second set of probes. In some embodiments, the first set of probes is configured to interrogate a region that is different in the target polynucleotides (i.e., a varying region). As set forth above, the varying region may have one or more bases that are different in the target polynucleotides (i.e., variable bases). The varying region may also have an identical sequence surrounding the variable bases. In some embodiments, the first set of probes has a region that can hybridize to both of the variable bases and surrounding bases. In some embodiments, the first set of probes has different affinity to each of the target polynucleotides. In some embodiments, the first set of probes has a sequence that is completely complementary to only one of target polynucleotides (e.g., the first target polynucleotide) but not the others (e.g., the second target polynucleotide). Taking a sequence of 5′-GAATAC-3′ (“-” means 0, 1 or more nucleotides are present) as an example, the underlined “C” is a variable base and the rest of “GAATA” is surrounding bases. In this example, the first target polynucleotide has “C” at the variable position whereas the second target polynucleotide has “A” in the same position. In one example, the first set of probes can have a sequence that is completely complementary to the first target polynucleotide such that the probes has 5′-GTATTC-3′ (the “G” complementary to the variable position is underlined). An alternative embodiment where the probes have a sequence that is completely complementary to the second target polynucleotide (i.e., 5′-TTATTC-3′ (the “T” complementary to the variable position is underlined)) is also possible. Therefore, in some embodiments, the first set of probes hybridize to the first target polynucleotide with a higher affinity than to the second target polynucleotide, or vice versa. In some embodiments, the first set of probes hybridize to only the first target polynucleotide but not to the second target polynucleotide, or vice versa. In these embodiments, signals indicative of this difference in hybridization are measured and processed to determine the genotype. In some other embodiments, the first set of probes has a sequence that is complementary to the surrounding region, but not to the variable base(s). In some embodiments, the first set of probes is designed to hybridize to a sequence that is 5′ or 3′ of the variable base(s). In some embodiments, the first set of probes hybridize to a sequence that is immediately 5′ or 3′ of the variable base(s). In some embodiments, the first set of probes terminates at a base immediately adjacent to the variable base(s). In some of these embodiments, the hybridization target (i.e., a specific target polynucleotide that hybridizes to each probe) can be distinguished via by incorporating a labeling molecule. For example, a differentially labelled nucleotide (e.g., A- or T-labelled with a first labeling molecule and G- or C-labelled with a second labeling molecule) can be incorporated to the probe depending on the target sequence that hybridizes to the probe via single-base extension or ligation, indicating the identity (or sequence) of the target polynucleotide.

It will be appreciated that genotyping can be carried out in any manner useful for the identification of varying sites in a plurality of target sequences of a nucleic acid sample. In some embodiments, methods of genotyping useful in connection with the present disclosure include those methods that are useful for SNP detection which is generally for analyzing alleles of a same gene. In some embodiments where two or more target genes are interrogated, the SNP for one or more target genes (e.g., a clinically relevant gene and/or its pseudogene) can be detected. Platforms for SNP detection are well known in the art and such platforms can be applicable in the methods provided herein for analyzing and interrogating two or more target sequences that are not from a same gene. Suitable methods for genotyping of the method herein include variations of single nucleotide extension, use of target-specific probes (e.g., probes that hybridize only to a single gene), ligation-based target discrimination, and the like.

In some embodiments, the array also contains a second set of probes that is configured to interrogate a region that is common or identical in the target polynucleotides. Therefore, the second set of probes hybridize to a region in which all of bases are invariant in the target sequences.

In examples where two target genes are interrogated by the methods provided herein, the second set of probes can be designed to hybridize to a region that is identical in both target genes. In some embodiments, the ‘region that is identical in both target genes’ refer to a sequence of nucleic acids that is identical in both genes when both genes are wild-type and do not have any mutations. In some cases, however, this region can be different between the two target genes in some individuals, if such individuals have mutation, deletion and/or insertion in their genome. In these examples, this region can still be interrogated with the second set of probes in order to determine the genotype and copy number of one or both target genes.

In some embodiments, the genotyping methods according to the present disclosure is configured to determine a combined copy number of the target polynucleotides in the nucleic acids of a sample. In some embodiments, this total copy number of the target polynucleotides is determined based on the hybridization profile of the second set of probes to the target polynucleotides. In some examples where the sample has two related genes (e.g., an actual gene and a pseudogene), the combined (or total) copy number of both genes are determined based on the signals indicative of the hybridization of the second set of probes to the nucleic acids in the sample. These signals, which is correlated to the abundance of both target genes, can be measured and normalized to signals from a reference sample. If the ratio of the signals between the test sample and the reference sample is different from an expected ratio, this may indicate variation of copy number for both genes. The signals of the reference may be signals measured from a sample that is known to be a normal diploid. The reference signals can be measured simultaneously with the test sample. Alternatively, the reference signals or data indicative of the reference signals can be provided, e.g., electronically. In some embodiments, there can be more steps to normalize other variable factors such as hybridization background and nucleic acid mass. In some embodiments, the measurement of the signals and processing the data associated with the measurement are processed via a certain algorithms, via a computer as described elsewhere in the present disclosure.

In some embodiments, the genotyping methods according to the present disclosure is configured to determine a ratio of the amounts between individual target polynucleotides. For example, if there are two target genes are interrogated for genotyping, the method determines the relative amount (i.e. ratio) of these two genes, e.g. 1:1, 2:0, 3:2 or more. This relative amounts of the target genes is determined based on the signals indicative of the hybridization of the first set of probes to the nucleic acids in the sample. These signals from the first probe set is correlated to the relative abundance of one target gene to the other in the nucleic acid sample. In some embodiments, the signals from the first target gene and the signals from the second target genes are measured and compared to each other to determine the ratio of the two genes. In some other embodiments, the ratio refers to an amount of one target gene relative to the total amount of both target genes. Therefore, in one example, the relative amount of the first target gene can be determined by dividing the signals from the first target gene by the sum of the signals from the first and second target gene. The relative amount of the second target gene can be determined by the same way except the signal from the second target gene is divided by the sum of the signals. In some embodiments, the relative amount of one target gene (e.g., the first target gene which is a clinically relevant gene such as SMN1) is used and sufficient to genotype and copy number determination. In some other embodiments, the relative amount of both target genes (e.g., a clinically relevant gene and its pseudogene such as SMN1 and SMN2) are utilized. In some embodiments, the measurement of the signals and processing the data associated with the measurement are processed via a certain algorithms, via a computer as described elsewhere in the present disclosure.

In the context of array based assays, a variety of genotyping methods are available. In some embodiments, the array surface is divided into features, each feature containing multiple sites that include copies of substantially identical oligonucleotides configured to bind to a particular target nucleic acid sequence. Hybridization of nucleic acid molecules to different locations on the array can be detected and quantified. One suitable method is to use any array containing target-specific probes that selectively bind only to a certain target(s) and not others. In other embodiments, the array contains probes that bind non-selectively to all of the different forms of target sequences, but then is extended or otherwise modified in a target-specific manner to generate a target-specific product. For example, the probe of the array can be elongated via template-dependent nucleotide polymerization. Alternatively, the probe can be elongated via sequence-dependent ligation of a tag oligonucleotide, which may contain a signal-generating moiety. In still, target-specific products (e.g., target-specific nucleotide extension products or ligation products) can be generated off-array, and then hybridized to an array containing probes that discriminate between the various extension products. Signals emitted from the array indicating hybridization of nucleic acid molecules to specific array probes can be detected and quantified. Examples of genotyping array products include the Affymetrix Axiom® arrays, the Affymetrix OncoScan arrays and the Affymetrix CytoScan arrays (Thermo Fisher Scientific) as well as Illumina's BeadChip® and Infinium® arrays. Suitable array-based genotyping methods are described, for example, in Hoffman et al, Genomics 98(2):79-89 (2011) and Shen et al., Mutation Research 573:70-82 (2005), both of which are incorporated herein in their entireties.

In some embodiments, the probes used in the methods provided herein have about 10 or more bases in length. In some embodiments, the probes have about 10 bases, about 20 bases, about 30 bases, about 40 bases, about 50 bases, about 50 bases, about 60 bases, about 70 bases, about 80 bases, about 90 bases, about 100 bases, about 200 bases, about 300 bases, about 400 bases, about 500 bases or any intervening number of bases of the foregoing in length. In some embodiments, the probes have 20 bases, 21 bases, 22 bases, 23 bases, 24 bases, 25 bases, 26 bases, 27 bases, 28 bases, 29 bases, 30 bases, 31 bases, 32 bases, 33 bases, 34 bases and 35 bases in length.

In some embodiments, the nucleic acids that are genotyped by the method of the disclosure include DNA and RNA obtained from a biological source (or biological sample) or an individual. The biological sample or source can be, for example, any number of tissues or fluids, such as blood, urine, serum, plasma, lymph, saliva, stool, and vaginal secretions, of virtually any organism. The nucleic acids for the genotyping can be genomic DNA, cell-free DNA and any types of RNA including mRNA.

In some embodiments, the nucleic acids interrogated by the method of the present disclosure is amplified and the amplified products are used for hybridization to the array. In embodiments where genomic DNA is used as a nucleic acid sample, the whole genome sequence can be amplified prior to hybridization to an array. In embodiments, the whole genome amplification is done via polymerase chain reaction (PCR) using random primers.

In some embodiments, the genotyping method according to the present disclosure includes a step of target amplification. In some embodiments, multiplex PCR (mPCR) is used to selectively amplify target genes. In some embodiments, among the target genes that includes a clinically relevant gene and its closely related pseudogene, only the clinically relevant gene or part thereof is selectively amplified. In some alternative embodiments, a plurality of target genes including a clinically relevant gene (or part thereof) as well as its related genes (or part thereof) are selectively amplified. In some embodiments, multiplex PCR products, which can be optionally diluted, are added to the nucleic acid sample, e.g., whole genomic DNA or amplified product thereof prior to the hybridization to the array. Alternatively or in combination, target polynucleotides are isolated using sequence-specific probes that are associated with a collectible means (e.g., biotin beads or antibodies). The sequence-specific probes that bind to the target sequence can be separated via pulling the biotin beads or antibodies using any suitable capture means (e.g., affinity chromatography).

In some embodiments, the genotyping method according to the present disclosure includes a step of fragmenting the nucleic acid sample or amplified products thereof. It will be appreciated that the fragmenting (or cleaving) can be accomplished according to any methods (e.g., physical methods such as shearing, sonication, heat treatment and more and chemical methods such as enzyme treatment) known in the art suitable for use in connection with the present disclosure. In some embodiments, one or more sequence-specific or sequence-nonspecific enzymes are used to fragment the nucleic acid sample or amplified products thereof. In some embodiments, one or more restriction enzymes can be used to fragment the nucleic acids for interrogation. In some embodiments, the step of fragmentation can be catalyzed by adding one or more enzymes, e.g., nucleases such as DNAase and/or a restriction enzyme. Suitable restriction enzymes include, but are not limited to, Aatll, Acc65I, Accl, Acil, AclI, Acul, Afel, AflII, AflIII, AgeI, AhdI, AleI, AluI, AlwI, AlwNI, ApaI, ApaLI, ApeKI, ApoI, AscI, AseI, AsiSI, AvaI, AvaII, AvrII, BaeGI, BaeI, BamHI, BanI, BanII, BbsI, BbvCI, BbvI, BccI, BceAI, BcgI, BciVI, BfaI, BfuAI, BfuCI, BglI, BglII, BlpI, BmgBI, BmrI, BmtI, BpmI, Bpul0I, BpuEI, BsaAI, BsaBI, BsaHI, Bsal, BsaJI, BsaWI, BsaXI, BscRI, BscYI, Bsgl, BsiEI, BsiHKAI, BsiWI, Bs1I, BsmAI, BsmBI, BsmFI, Bsml, BsoBI, Bsp12861, BspCNI, BspDI, BspEI, BspHI, BspMI, BspQI, BsrBI, BsrDI, BsrFI, BsrGI, BsrI, BssHII, BssKI, BssSI, BstAPI, BstBI, BstEII, BstNI, BstUI, BstXI, BstYI, BstZ17I, Bsu36I, BtgI, BtgZI, BtsCI, BtsI, Cac8I, ClaI, CspCI, CviAII, CviKI-1, CviQI, DdcI, DpnI, DpnII, DraI, DraIII, DrdI, EacI, EagI, EarI, EciI, Eco53kI, EcoNI, EcoO109I, EcoP15I, EcoRI, EcoRV, FatI, FauI, Fnu4HI, FokI, FseI, FspI, HaeII, HaeIII, HgaI, HhaI, HincII, HindIII, HinfI, HinPlI, HpaI, HpaII, HphI, Hpyl66II, Hpy188I, Hpy188III, Hpy99I, HpyAV, HpyCH4III, HpyCH4IV, HpyCH4V, Kasl, Kpnl, Mbol, Mboll, MfeI, MluI, MlyI, MmeI, Mn1I, MscI, MseI, Ms1I, MspA1I, MspI, MwoI, NaeI, NarI, Nb.BbvCI, Nb.BsmI, Nb.BsrDI, Nb.BtsI, NciI, NcoI, NdeI, NgoMIV, NheI, NlaIII, NlaIV, NmeAIII, NotI, NruI, NsiI, NspI, Nt.AlwI, Nt.BbvCI, Nt.BsmAI, Nt.BspQI, Nt.BstNBI, Nt.CviPII, PacI, PaeR7I, PciI, PflFI, PflMI, Phol, Plel, Pmel, Pm1I, PpuMI, PshAI, Psil, PspGI, PspOMI, PspXI, Pstl, Pvul, Pvull, Rsal, Rsrll, Sad, SacII, SalI, SapI, Sau3AI, Sau96I, Sbfl, ScaI, ScrFI, SexAI, SfaNI, SfcI, SfiI, SfoI, SgrAI, SmaI, Smll, SnaBI, Spel, Sphl, Sspl, Stul, StyD4I, Styl, Swal, T, TaqαI, Tfil, TliI, Tsel, Tsp45I, Tsp509I, TspMI, TspRI, Tth111I, Xbal, Xcml, Xhol, Xmal, Xmnl, and Zral. In some embodiments, the fragmented nucleic acids or amplified products thereof are provided to an array for genotyping.

In some embodiments, the methods described in the present disclosure include a step of genotyping. The genotyping can include determining the sequence of at least one nucleotide within a target nucleic acid sequence. In some embodiments, the step of genotyping involves analyzing a plurality (e.g., one, two or more) target polynucleotides from a sample, which may be obtained from a biological source or organism. In some embodiments, the target polynucleotides are different genes. In some embodiments, the target nucleic acids include a clinically relevant gene and other nucleic acid sequence(s) that share some sequence identity, e.g., one or more related genes such as pseudogenes. In some embodiments where two or more target genes are interrogated, the methods described herein are used to genotype one of the target genes such as a clinically relevant gene or genes. In some embodiments, the methods described herein are used to genotype a clinically not (or less) relevant gene or genes. In some embodiments, the methods described herein are used to genotype both the clinically relevant gene(s) and its related, clinically not (or less) relevant gene(s).

In one aspect, the disclosed herein provides a computer-implemented method for genotyping a mixture of nucleic acids. The mixture may have a first target polynucleotide and a second target polynucleotide having a sequence identity of at least 50% to the first target polynucleotide. The method may include obtaining, by a computer comprising a processor, first data of an intensity measurement from a first set of probes, obtaining, by the computer, second data of an intensity measurement from a second set of probes, and determining, by the processor, from the first data a ratio of the first and second target polynucleotides in the mixture. The method then determines from the second data a combined copy number of the first and second target polynucleotides in the mixture through operation of the processor. The method then determines a genotype of at least one of the first and second target polynucleotides, through operation of the processor.

In some embodiments, the first set of probes targets a sequence that is different in the first and second target polynucleotide sequences and the second set of probes targets a sequence that is identical in the first and second target polynucleotides sequences.

In some embodiments, the first and second sets of probes may be provided in an array. The first and second sets of probes may hybridize to the target polynucleotides on the array. The nucleotide sequences may be from a human.

In some embodiments, the ratio of the first and second target polynucleotides may be a ratio of the first and second target polynucleotides in a human genome. The combined copy number of the first and second target polynucleotides may be a combined genomic copy number of the first and second target polynucleotides in a human genome.

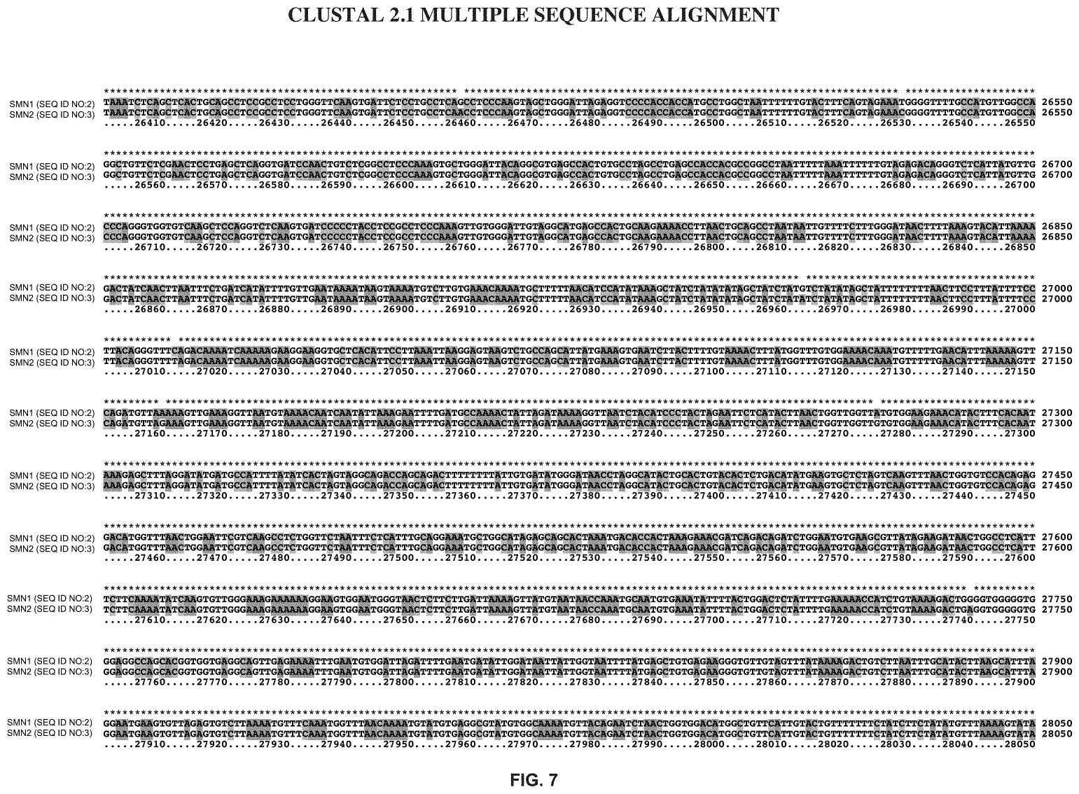

In some embodiments, the first and second target polynucleotides are from different genes. The first and second target polynucleotides may also not be allelic variants of the same gene. The target polynucleotides may correspond to Survival Motor Neuron 1 (SMN1) and Survival Motor Neuron 2 (SMN2) genes or part thereof. The first target polynucleotide may be found in the SMN2 gene and a variant of SMN1 gene with a mutation in and around exon 7. The second target polynucleotide may be found in the SMN1 gene. Alternatively, the second target polynucleotide may be found in the SMN2 gene and a variant of SMN1 gene with a mutation in and around exon 7 and the first target polynucleotide may be found in the SMN1 gene. In some embodiments, the first set of probes may include at least four probe sets and each probe set corresponds to a sequence that is different in SMN1 and SMN2 genes. In some embodiments, the at least four probe sets targeting variants in and around exon 7 of the SMN1 gene target the following regions: a region containing chromosome 5:70,247,773C>T site (position 27,012 in FIG. 7), a region containing chromosome 5: 70,247,921A>G site (position 27,160 in FIG. 7), a region containing chromosome 5: 70,248,036A>G site (position 27,275 in FIG. 7), and a region containing chromosome 5: 70,248,501G>A (position 27,740 in FIG. 7). In some embodiments, the probe sets can also include one or more probes targeting a polymorphic region or site of SMN1. For example, a region containing g.27134T>G site (chromsome5: 70,247,901, position 27,134 in FIG. 7), which is genetically linked to a silent carrier mutation for SMN1 can be used. In some embodiments, the copy-number of SMN1 can be called via the doubly normalized depth at the single intronic base that distinguishes SMN1 and SMN2. When calling the chromosome 5:70,247,773C>T SNP in SMN1, only those fragments containing the SMN1-distinguishing intronic base may populate the pileup of reads used to call chromosome 5:70,247,773C>T, and the copy-number of SMN1 may define the expected allele balances to consider (e.g., with three copies of SMN1, an allele balance of 0%, 33%, 66%, or 100% is expected). All genomic locations cited above are in GRCh37/hg19 coordinates.

In some embodiments, the method involves receiving data of signals from the array. A first target polynucleotide may be reported by the first set of probes. An average intensity value for the probe sets may be calculated as well as a determination for a standard deviation between the average intensity values. The method may calculate a raw frequency of the target polynucleotides. The raw frequency may be utilized in the calculation of a centered frequency of the target polynucleotides. The centered frequency may be utilized to calculate a scaled and centered frequency of the target polynucleotides. A median frequency of the target polynucleotides may be calculated from an affinity value of each probe set for the target polynucleotides and predicted copy number (CN). Hyperplanes may be delineated from the data corresponding to presence of no copy of the target polynucleotides in the mixture, one copy of the target polynucleotides gene in the mixture, and two copies of the target polynucleotides in the mixture. A quantity of probe set clusters within the hyperplanes may then be correlated as a statistical indication of the number of copies of the target polynucleotides in the mixture.

In some embodiments, the method may perform scaling operation to further scale the scaled and centered frequency by setting the scaled and centered frequency to 1 in response to the scaled and centered frequency being greater than 1. The scaling operation may also set the scaled and centered frequency to 0 in response to the scaled and centered frequency being less than 0. The scaling operation may then determine the direction of the frequency by subtracting a median frequency for the first target polynucleotide, and using the median frequency value for the second target polynucleotide.

In some embodiments, the calculation of the raw frequency for the probe sets may include dividing an intensity for the second target polynucleotide by the sum of an intensity for the first target polynucleotide and an intensity for the second target polynucleotide. In some embodiments, this calculation is done with the data obtained from the first set of probes. In some embodiments, this calculation is done with the data obtained from the second set of probes.

In some instances, the calculation of the raw frequency for the probe sets include dividing an intensity for the first target polynucleotide by the sum of an intensity for the first target polynucleotide and an intensity for the second target polynucleotide. In some embodiments, this calculation is done with the data obtained from the first set of probes. In some embodiments, this calculation is done with the data obtained from the second set of probes.

In some embodiments, the calculation of the centered frequency for the probe sets from the raw frequency further may involve subtracting the standard deviation from the raw frequency and then adding an ideal frequency ratio of 0.5, the ideal frequency being the frequency between the first and second target polynucleotides.

In some embodiments, the calculation of the scaled and centered frequency for the probe sets from the centered frequency may involve multiplying the difference between the centered frequency and a first alpha cutoff value by a first scaling factor and then subtracting this value from the first alpha cutoff value, in response to the centered frequency being less than the first alpha cutoff value. The difference between the centered frequency and a second alpha cutoff value may be multiplied by a second scaling factor and then adding this value to the second alpha cutoff value, in response to the centered frequency being greater than the second alpha cutoff value. The centered frequency may be identified as the scaled and centered frequency, in response to the centered frequency being equal or within a range formed by the first alpha cutoff value and the second alpha cutoff value.

In some embodiments, the method involves plotting the scaled and centered frequency for the probe sets against their predicted copy number on a graph. Hyperplanes may then be delineated in the graph corresponding to presence of no copy of the target polynucleotides in the mixture, one copy of the target nucleotides in the mixture, and two copies of the target nucleotides in the mixture. The quantity of probe set clusters within the hyperplanes may then be correlated with the statistical indication of the number of copies of the target nucleotides in the mixture.

In some embodiments, the method involves normalizing the raw frequency for the probe sets. In some embodiments, the normalization of the raw frequency for each of the probe sets may involve calculating a centered frequency for the probe sets from the raw frequency by subtracting the standard deviation from the raw frequency and then adding an ideal frequency ratio of 0.5, the ideal frequency being the raw frequency between the first and second target polynucleotides. In some embodiments, the normalization may also involve calculating a scaled and centered frequency for each of the probe sets from the centered frequency. In some embodiments, the calculation of the scaled and centered frequency may involve multiplying the difference between the centered frequency and a first alpha cutoff value by a first scaling factor and then subtracting this value from the first alpha cutoff value, in response to the centered frequency being less than the first alpha cutoff value. In some embodiments, the calculation of the scaled and centered frequency may involve multiplying the difference between the centered frequency and a second alpha cutoff value by a second scaling factor and then adding this value to the second alpha cutoff value, in response to the centered frequency being greater than the second alpha cutoff value. In some embodiments, the calculation of the scaled and centered frequency may also involve identifying the centered frequency as the scaled and centered frequency in response to the centered frequency being equal or within a range formed by the first alpha cutoff value and the second alpha cutoff value.

### Carrier Screening

In some embodiments, the disclosure provided herein is useful in the diagnosis of a carrier status of an individual for a pathological condition or disease. For example, the methods, compositions, kits, systems, devices and instruments provided herein are useful in determining if an individual can be a carrier for an autosomal recessive disease such that a risk for a child of the individual of being affected by the disease can be accessed.

Autosomal recessive inheritance is a condition that appears only in individuals who have received two copies of an altered gene, one copy from each parent. The parents are carriers who have only one copy of the gene and do not exhibit the trait because the gene is recessive to its normal counterpart gene. As illustrated in FIG. 1, if both parents are carriers, there is a 25% chance of a child inheriting both abnormal genes and, consequently, developing the disease. There is a 50% chance of a child inheriting only one abnormal gene and of being a carrier, like the parents, and there is a 25% chance of the child inheriting both normal genes.

A hereditary carrier (or simply a carrier), is a person or other organism that has inherited a recessive allele for a genetic trait or mutation but may not display that trait or show symptoms of the disease. Carriers are able to pass the allele onto their offspring, who may then express the genetic if they inherit the recessive allele from both parents. The chance of two carriers having a child with the disease is 25%.

There are a number of diseases or conditions that are determined by autosomal recessive inheritance. Some examples include cystic fibrosis, sickle cell anemia, fanconi anemia, pyruvate dehydrogenase deficiency, Xeroderma pigmentosum, Hartnup's disease, Kartagener's Syndrome, Tay-Sachs disease and spinal muscular atrophy (SMN). While diagnosis of these diseases or conditions (i.e., determining if an individual is a patient of the disease or condition or has a risk of being affected) is of paramount importance, it is also important to screen an individual, who is planning to have a child soon or later, and determine if the individual is a carrier for the disease or condition. Such screening can be particularly useful, for example, in the course of in-vitro fertilization (IVT).

In some embodiments, the disclosure herein provides a method of determining a carrier status of an individual for an autosomal recessive condition. The method may include a step of providing nucleic acids obtained from the individual or amplified products thereof to an array. The array may have a first set of probes and a second set of probes that hybridize to a first target polynucleotides and a second target polynucleotide. The first set of probes hybridizes to a first region that has a sequence different in the first and second target polynucleotides and the second set of probes hybridize to a second region that is identical in the first and second target polynucleotides. The first and second genes may have sequence identity of at least 50%. The method may include a step of detecting a signal indicative of the hybridization of the first set of probes to the nucleic acids of the individual or the amplified products thereof. The method may also include a step of detecting a signal indicative of the hybridization of the second set of probes to the nucleic acids of the individual or the amplified products thereof. The method may further include a step of genotyping the nucleic acids of the individual by analyzing the signals and determining the carrier status of the individual based on the genotype.

In some embodiments, the first region that is interrogated by the method for carrier screening provided herein has one or more base(s) that are different (variable) in the target polynucleotides and a sequence that is near or surrounding the variable base(s). In some embodiments, the first set of probes hybridizes to a sequence that is immediately 5′ or 3′ of the variable base(s). In some embodiments, the first set of probes terminates at a base immediately adjacent to the varying base(s). In some embodiments, the first set of probes includes a sequence that is complementary to the variable base(s).

In some embodiments, the target polynucleotides that are interrogated by the method for carrier status herein are from different genes. In some embodiments, the target polynucleotides are not allelic variants of a gene. In some embodiments, the method interrogates at least two genes, e.g., a clinically relevant gene and its related gene (e.g., a pseudogene). One example of such a pair of genes include Survival Motor Neuron 1 (SMN1) and SMN2 genes. The method provided herein therefore can be used to screen a carrier for spinal muscular atrophy (SMA) that is associated with SMN 1 gene.

In some embodiments, the method of determining a carrier status provided herein further includes a step of determining a combined copy number of the first and second target polynucleotides in the nucleic acids of the individual. In some embodiments, the method also includes determining a ratio of the amounts of the first and second target polynucleotides in the nucleic acids of the individual. In some embodiments, the method also includes determining an amount of a target polynucleotide relative to the total amount of the total target nucleotides. Therefore, for example, the relative amount of a first target polynucleotide can be determined by dividing the signals from the first target polynucleotide by the sum of signals from the first and second target polynucleotides. The relative amount of the second target polynucleotide can be determined in the same way except the signals from the second target polynucleotide is divided by the sum of the signals.

In some embodiments, the target polynucleotides interrogated by the carrier screening method provided herein have sequence identity of at least about 50%, about 55%, about 60%, about 65%, about 70%, about 75%, about 80%, about 85%, about 90%, about 95%, about 99%, about 99.99% or any intervening percentage of the foregoing.

In some embodiments, the nucleic acids interrogated by the carrier screening method herein has genomic DNA obtained from an individual. In some other embodiments, other types of nucleic acids such as floating DNA (e.g., cell-free DNA) or RNA (e.g., mRNA, siRNA or miRNA) can be used as a sample of nucleic acids for the method.

In some embodiments, the method of determining a carrier status provided herein further includes a step of amplifying target polynucleotides. This amplification step can include amplifying nucleic acids of the target polynucleotides. As described elsewhere in the disclosure, the amplification can be done via, e.g., a polymerase chain reaction (PCR) with sequence-specific primers. Alternatively or in combination, target polynucleotides are isolated using sequence-specific probes that are associated with a collectible means (e.g., biotin beads or antibodies). The sequence-specific probes that bind to the target sequence can be separated via pulling the biotin beads or antibodies using any suitable capture means (e.g., affinity chromatography).

In some embodiments, the method of determining a carrier status provided herein further includes a step of fragmenting nucleic acids obtained from an individual or amplified products thereof, thereby generating fragmented nucleic acids. This fragmentation can be done according to any method known in the art suitable for use in connection with the present disclosure. In some embodiments, one or more sequence-specific or sequence-nonspecific enzymes are used to fragment the nucleic acid sample or amplified products thereof. In some embodiments, one or more restriction enzymes can be used to fragment the nucleic acids. In some embodiments, the step of fragmentation can be catalyzed by adding one or more enzymes, e.g., nucleases such as DNAase or a restriction enzyme. In some embodiments, two or more enzymes can be used to fragment the nucleic acids or amplified products thereof. In some embodiments, the fragmented nucleic acids or amplified products thereof are provided to an array for carrier status screening.

In some embodiments, the method of determining a carrier status provided herein further includes a step of determining the presence or absence of mutations, insertions, and/or deletions in a target polynucleotide (e.g., a gene that is clinically relevant) so as to determine the presence or absence of a functional copy of the target polynucleotide in the individual. The functional copy of a gene may refer to a gene copy that has at least about 30% of the activity of the wild-type copy of the gene. In some embodiments, the functional copy of a gene include a gene copy that has at least about 35%, about 40%, about 45%, about 50%, about 55%, about 60%, about 65%, about 70%, about 75%, about 80%, about 85%, about 90%, about 95%, about, 97%, about 99%, about 100% of the activity of the wild-type copy of the gene or any intervening percentage of the foregoing. Various methods of determining the functionality (or activity) of a gene copy are available in the art. For example, there are various computational prediction methods available in the field, e.g., the Virtual Gene Oncology (VIRGO) service (Naveed Massjouni, Corban Rivera, and T. M. Murali, VIRGO: computational prediction of gene functions, Nucleic Acids Rsearch (2006), vol. 34, pages W340-W344) and SynFPS system (Jason Li, Saman Halgamuge, Christopher Kells, and Sen-Lin Tang, Gene function prediction based on genomic context clustering and discriminative learning: an application to bacteriophages, BMC Bioinformatics (2007), 8 (Suppl 4): S6, which are incorporated in their entireties herewith). In addition, various experimental approaches are available in the field to test and/or measure the functionality of a specific form of a gene including enzyme activity assay, binding affinity assay, reporter-based assay or complementation assay and many others. Therefore, in some embodiments, once the structure of a specific copy of the target gene in a test sample is analyzed with the method provided herein, one can computationally predict or experimentally test the functionality (or activity) of the specific copy of the gene.

In some embodiments, the method of determining a carrier status provided herein further includes a step of determining if an individual is a carrier for the autosomal recessive condition of interest. In some embodiments, the individual is determined to be a carrier if the copy number of a target polynucleotide (e.g., a gene that is clinically relevant for the condition of interest) from the individual is 1. In some embodiments, the individual is determined to be a carrier if he or she has one functional copy of the target gene, e.g., the copy that has at least about 30% to about 100% of functionality of the wild-type target gene. In some embodiments, the tested individual has two or more copies of the target gene in which only one copy is a functional copy and the other(s) is(are) non-functional copy(ies) of the target gene. In such a case, the tested individual may still be considered a carrier for having only one functional copy of the target gene.

In another aspect, the disclosure herein provides a method of operating a carrier detection algorithm, which may involve receiving a probe set data for an array having a first set of probes and a second set of probes, the first set of probes targeting a variable sequence of first and second target polynucleotides and the second set of probes targeting an identical sequence of the target polynucleotides, the data comprising an average signal intensity for the target polynucleotide to each probe sets, a standard deviation of the average signal intensity for each probe set, a first scaling factor, a second scaling factor, and a copy number region. In some embodiments, the method involves calculating a raw frequency of one or both of the target polynucleotides from the average signal intensity from the probe sets. In some embodiments, a centered frequency of the target polynucleotides may be calculated from the respective raw frequency, an ideal frequency ratio, and the standard deviation. In some embodiments, a scaled and centered frequency of the target polynucleotides is calculated from the respective centered frequency, a first alpha cutoff value, a second alpha cutoff value, the first scaling factor, and the second scaling factor. In some embodiments, a median frequency of the target polynucleotides is calculated from an affinity value of each probe set for the target polynucleotides and predicted copy number (CN). In some embodiments, hyperplanes are delineated corresponding to presence of no copy of the target polynucleotides, one copy of the target polynucleotides, and two copies of the target polynucleotides. In some embodiments, a quantity of probe set clusters within the hyperplanes is correlated to a statistical indication of the number of copies of the target polynucleotides. In some instances, the target polynucleotides are human sequences.

In some embodiments, the copy number of the target polynucleotides may be a genomic copy number of the target polynucleotides in a human genome. The first and second target polynucleotides may have sequence identity of at least 50%. In some embodiments, the first and second target polynucleotides are from different genes. In some embodiments, the first and second target polynucleotides are not allelic variants of a gene.

In some embodiments, the target polynucleotides may be Survival Motor Neuron 1 (SMN1) and Survival Motor Neuron 2 (SMN2) genes or part thereof. In some embodiments, the first target polynucleotide is found in the SMN2 gene and a variant of SMN1 gene with a mutation in and around exon 7. In some embodiments, the second target polynucleotide is found in the SMN1 gene. Alternatively, the second target polynucleotide is found in the SMN2 gene and a variant of SMN1 gene with a mutation in and around exon 7, and the first target polynucleotide is found in the SMN1 gene. The first set of probes may include at least four probe sets and each probe set corresponds to a sequence that may be different in SMN1 and SMN2 genes.

In some embodiments, the at least four probe sets targeting variants in and around exon 7 of the SMN1 gene target the following regions: a region containing chromosome 5:70,247,773C>T site, a region containing chromosome 5: 70,247,921A>G site, a region containing chromosome 5: 70,248,036A>G site, and a region containing chromosome 5: 70,248,501G>A.

In some embodiments, the scaled and centered frequency is scaled by setting the scaled and centered frequency to 1, in response to the scaled and centered frequency being greater than 1. In some embodiments, the scaled and centered frequency is scaled by setting the scaled and centered frequency to 0 in response to the scaled and centered frequency being less than 0. In some embodiments, the method then involves determining the direction of the raw frequency by subtracting a median frequency value for the first target polynucleotide and using the median frequency value for the second target nucleotide.

In some instances, calculating the raw frequency for the probe sets involves dividing an intensity for the second target polynucleotide, by the sum of an intensity for the first target polynucleotide and the intensity for the second target polynucleotide.

In some embodiments, calculating the raw frequency for the probe sets involves dividing an intensity for the first target polynucleotide by the sum of the intensity for the first target polynucleotide and an intensity for the second target polynucleotide.

In some embodiments, the calculation of the centered frequency for the probe sets from the raw frequency involves subtracting the standard deviation from the raw frequency and then adding the ideal frequency ratio of 0.5, the ideal frequency being the raw frequency between the first and second target polynucleotides.

In some embodiments, calculating the scaled and centered frequency for each of the probe sets from the centered frequency involves multiplying the difference between the centered frequency and the first alpha cutoff value by the first scaling factor and then subtracting this value from the first alpha cutoff value, in response to the centered frequency being less than the first alpha cutoff value. In some embodiments, the calculation of the scaled and centered frequency for each of the probe sets from the centered frequency involves multiplying the difference between the centered frequency and the second alpha cutoff value by the second scaling factor and then adding this value to the second alpha cutoff value, in response to the centered frequency being greater than the second alpha cutoff value. In some embodiments, the calculation of the scaled and centered frequency for each of the probe sets from the centered frequency also involves identifying the centered frequency as the scaled and centered frequency, in response to the centered frequency being equal or within a range formed by the first alpha cutoff value and the second alpha cutoff value.

In some embodiments, the method involves plotting the scaled and centered frequency for the target polynucleotides against their predicted copy number on a graph. In some embodiments, the method then delineates the hyperplanes in the graph corresponding to presence of no copy of the target polynucleotides, one copy of the target polynucleotides, and two copies of the target polynucleotides. In some embodiments, the method then correlates the quantity of probe set clusters within the hyperplanes as the statistical indication of the number of copies of the target polynucleotides in a human genome.

In another aspect, the disclosure herein provides methods of identifying a carrier genotype of an autosomal recessive condition in a subject. The method may involve obtaining first data for a first set of probes targeting a first marker sequence that is different in a first polynucleotide sequence and a second polynucleotide sequence, wherein the first and second polynucleotide sequences may have sequence identity of at least 50% and the autosomal recessive condition may be caused at the absence of functional copies of first polynucleotide sequence in a genome. The method may also involve obtaining second data for a second set probes targeting a second marker sequence that may be identical in the first polynucleotide sequence and the second polynucleotide sequence. A copy number for at least one polynucleotide sequences and a ratio identifying relative presence of the first and second polynucleotide sequences may be calculated from the first data and the second data. A carrier genotype may be identified when the copy number of the first polynucleotide sequence is less than 2 and/or when the ratio indicates a higher presence of the second polynucleotide sequence relative to the first polynucleotide sequence.

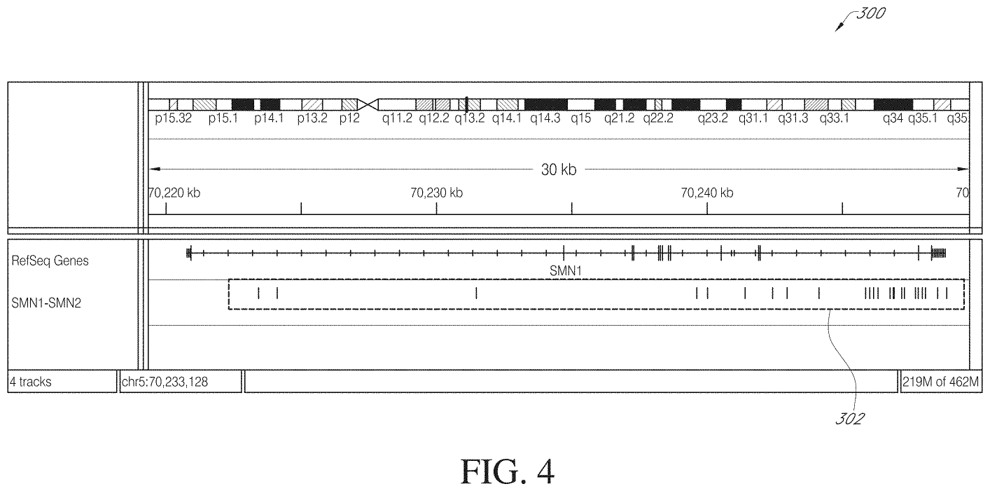

In some embodiments, the methods of identifying a carrier genotype provided herein is useful to access a risk for SMA, which is caused by autosomal inheritance of SMN1. Human genome sequence has SMN2 which is highly similar in sequence to SMN1. FIG. 4 illustrates genome browser 300 showing the alignment of SMN2 to SMN1 set as the reference sequence. The genome browser 300 shows markers 302 identifying 26 variant locations in 28 kilo bases invariant in each gene.

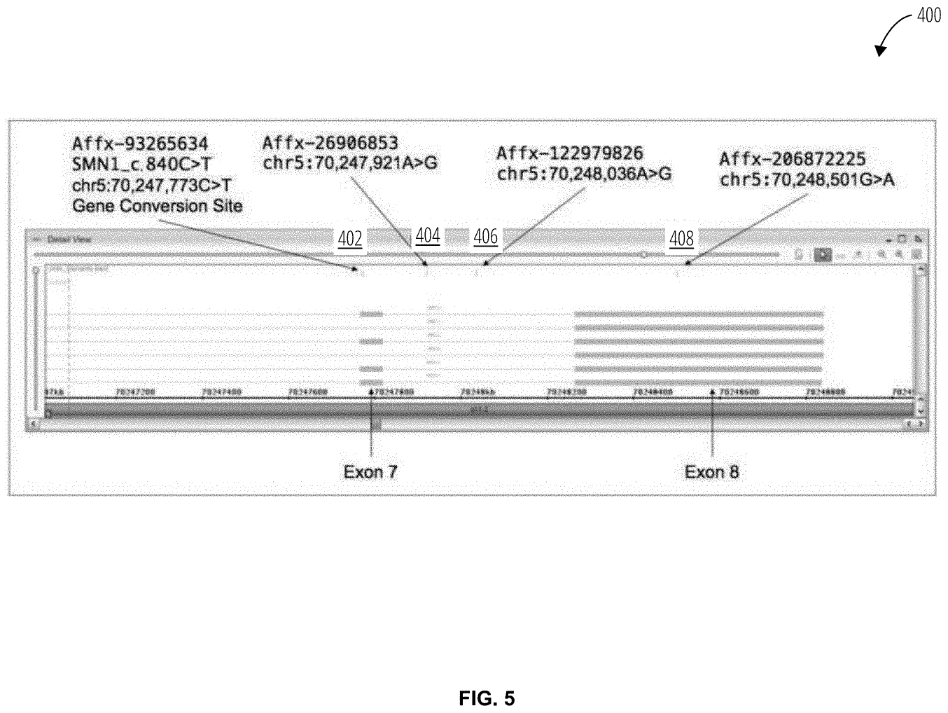

Referencing FIG. 5, a genomic browser 400 illustrates an enhanced view comparing the exon 7 of SMN1 and SMN2. Within the region of exon 7 the four markers are present. Marker 402 identifies the gene conversion site that differentiates a functioning copy of SMN1 from SMN2. Marker 402 is found at chr5: 70,247,773 and is a C>T conversion. The marker 402 also indicates the common carrier variant of SMN1. Marker 404 is another point mutation differentiating SMN1 from SMN2. Marker 404 is found at chr5: 70,247,921 and is an A>G conversion. Marker 406 is another point mutation differentiating SMN1 from SMN2. Marker 406 is found at Chr5: 70,248,036 and is a A>G conversion. Marker 408 is another point mutation differentiating SMN1 from SMN2. Marker 408 is found at Chr5: 70,248,501 and is a G>A conversion.

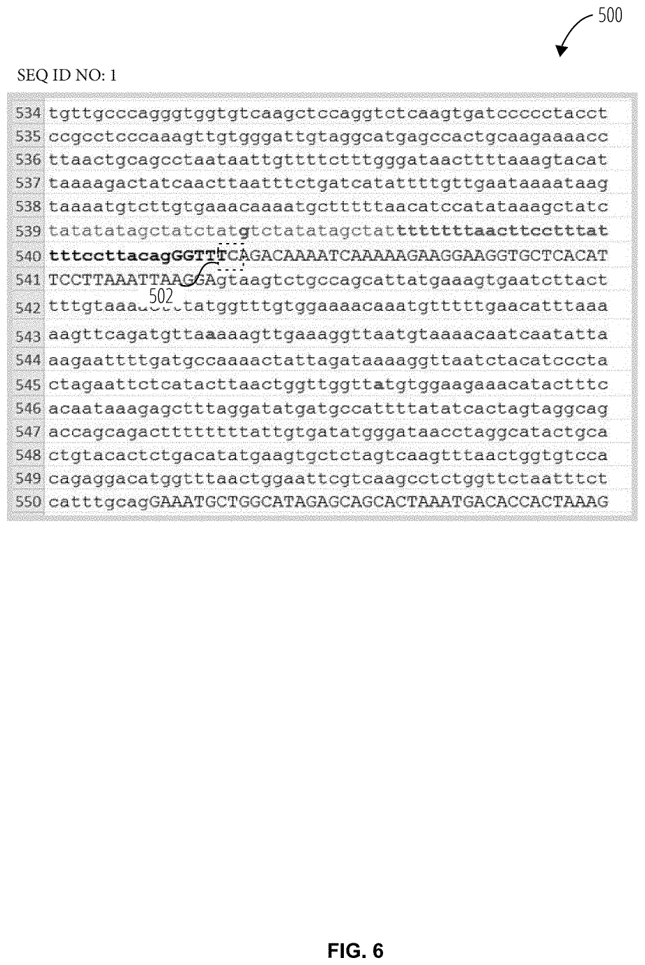

FIG. 6 illustrates an SMN1 base sequence 500. Lower case blue bases are SMN1-specific. Exon 7 has 54 base pairs (shown in upper case). Exon 7 SNP shown as red C (marker 502) indicates the gene conversion site that appears as a T in SMN2. Allele-specific primers may be designed to target sequence these differences, for use in assessing amplicon size and intensity as a function of SMN1 copy number (CN).

In some embodiments, one or more primer sets are utilized to prepare amplicons SMN1 and/or SMN2. Each primer has four different mismatch designs, resulting in a total of 64 different primer combinations to test. In some embodiments, only SMN1 or part thereof is amplified. Alternatively, both of SMN1 and SMN2 or parts thereof are amplified.

FIG. 7 illustrates a sequence alignment between a region of SMN1 and a corresponding of SMN2 upstream of exon 7. The sequence alignment shows the alignment of the sequence upstream of the exon 7 region of the two genes. The variations between the two sequences can be utilized to differentiate the two genes.

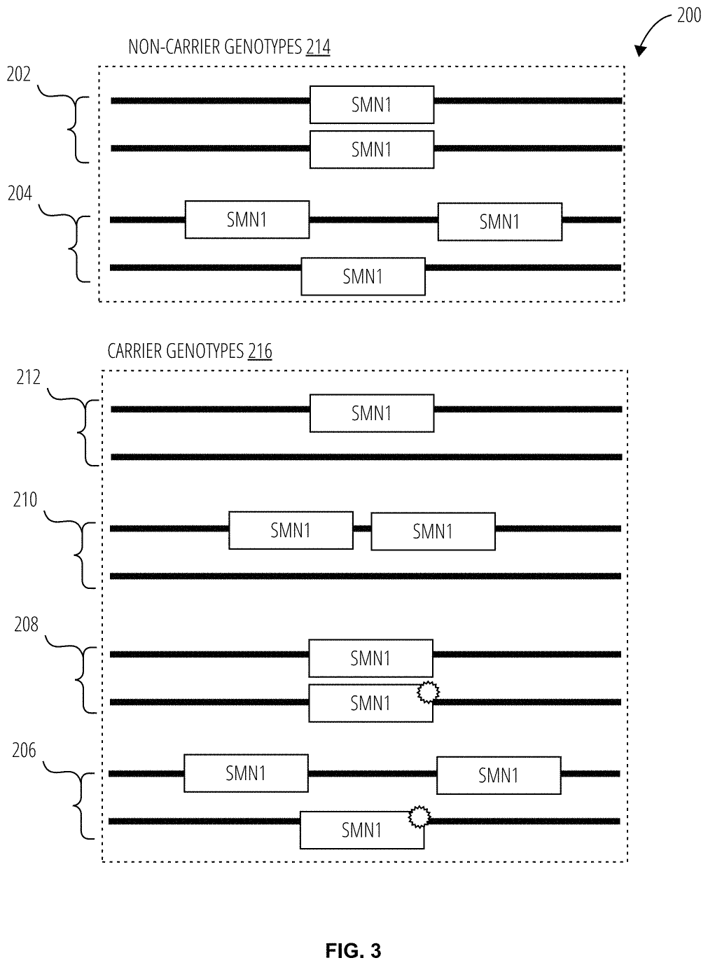

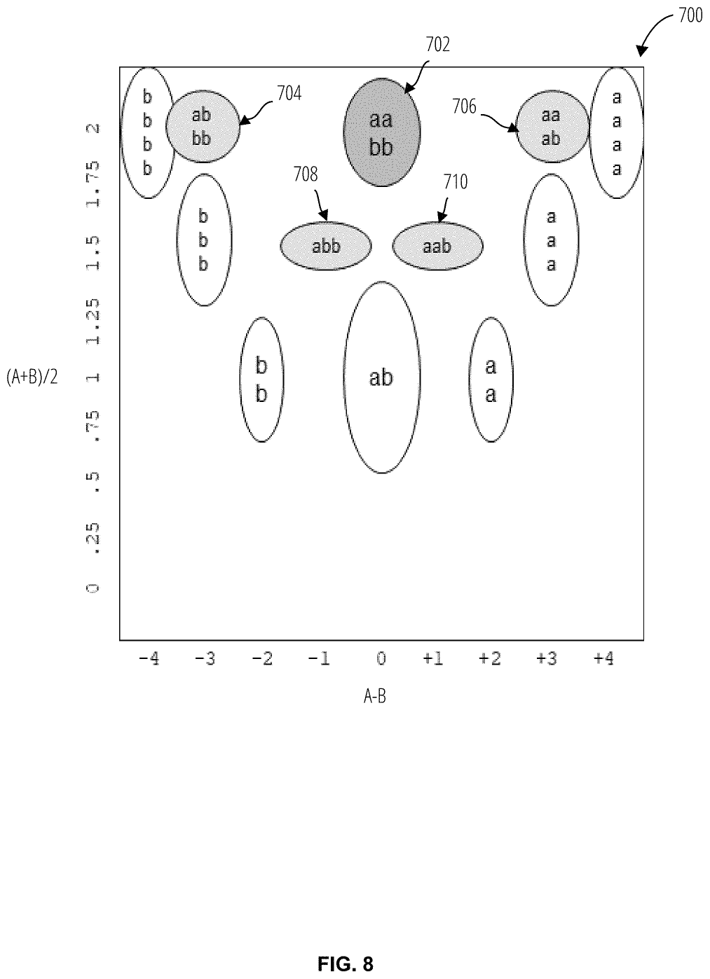

FIG. 8 illustrates selected SMN1-SMN2 sequence variant genotypes 700. SMN1 and SMN2 have nearly identical sequences and will behave similar to a tetraploid. The selected variants are non-polymorphic in SMN1 and SMN2, and thus a typical sample will be ‘aabb’ and fall in the normal cluster 702. “a” and “b” herein represents copy of SMN1 and SMN2, respectively. The normal cluster 702 includes non-carrier genotypes 214 such as ‘1+1’ genotype 202 where SMN1 is found on both copies, and ‘2+1’ genotype 204 where one of the DNA strands include two working version of the SMN1 gene (see FIG. 3). Both non-carrier genotypes 214 meet the requirement of having at least one working copy of the SMN1 gene on each DNA strand. Carrier genotypes 216 differ from the non-carrier genotypes 214 by the having at least one DNA strand without a working copy of SMN1 gene. For instances ‘1+0’ genotype 212 is a carrier where the SMN1 gene is missing from one of the DNA strands, or ‘1+1*’ genotype 208 where one of the DNA strands includes a non-functioning copy of the SMN1 gene. These particular genotype are considered a common carrier. Unlike ‘1+0’ genotype 212 and ‘1+1*’ genotype 208, ‘2+0’ genotype 210 is known as a silent carrier as it able to function similarly to a non-carrier genotype, in terms of protein production, but lacks a SMN1 gene on one of the DNA strands, resulting in 50% of the gametes having no of the SMN1 gene. Similarly, a ‘2+1*’ genotype 206 shares the duplicate gene on the same DNA strand, but lacks a working copy of SMN1 on the other.

Depending on the probes utilized, the ‘1+1*’ genotype 208 (see FIG. 3) with the mutated SMN1 could fall in the variant cluster 704 or variant cluster 706 with a copy number of 4. The ‘1+0’ genotype 212 with the deleted SMN1 would falls in the variant cluster 708 or variant cluster 710 as it would have a copy number of 3.

In some embodiments, the system detects genotypes in the variant clusters along with the copy number of the SMN1 and SMN2 genes. The genotype clusters identify various copy number and genotypes. The system may aggregate data over the (e.g., 26) variants to build a consensus on the number of SMN1 and SMN2 genes. An expectation may be set, for example that 1 in 50 samples will be from carriers, and therefore for example approximately two samples per analysis plate will identify as carriers. The samples should comprise a high replicate count to ensure the clusters are “tight” (low spread). The system should, on average, detect one or two samples outside of main cluster (normal cluster 702).

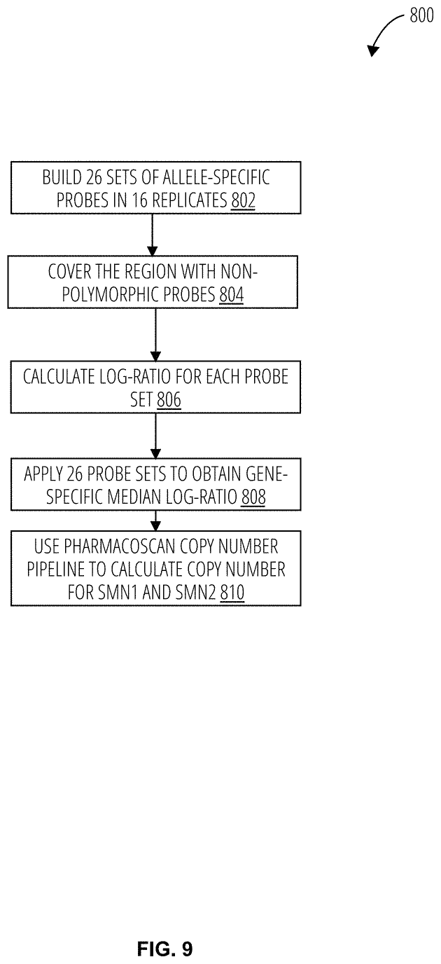

FIG. 9 illustrates an embodiment of a copy number determination process 800. Based on 26 gene specific nucleotides, build 26 sets of allele-specific probes in 16 replicates (block 802). Also cover the region with non-polymorphic probes (block 804). Calculate a log-ratio for each probe set (block 806)

In some embodiments, the log ratio is computed using non-polymorphic probes.

In some embodiments, gene-specific median log-ratios are calculated from non-polymorphic probes to calculate copy number for SMN1 and SMN2 (block 804).

In some embodiments, the log ratio computation usually avoids probe that map to more than one location in the genome. In one embodiment shown in FIG. 9, the probes are selected to get a “combined” copy number for SMN1 and SMN2 genes. In some embodiments, the combined copy number for SMN1 and SMN2 genes means the combined genomic copy number for both genes in the genome of the source, e.g., the genome of the individual from which the nucleic acid sample was obtained.

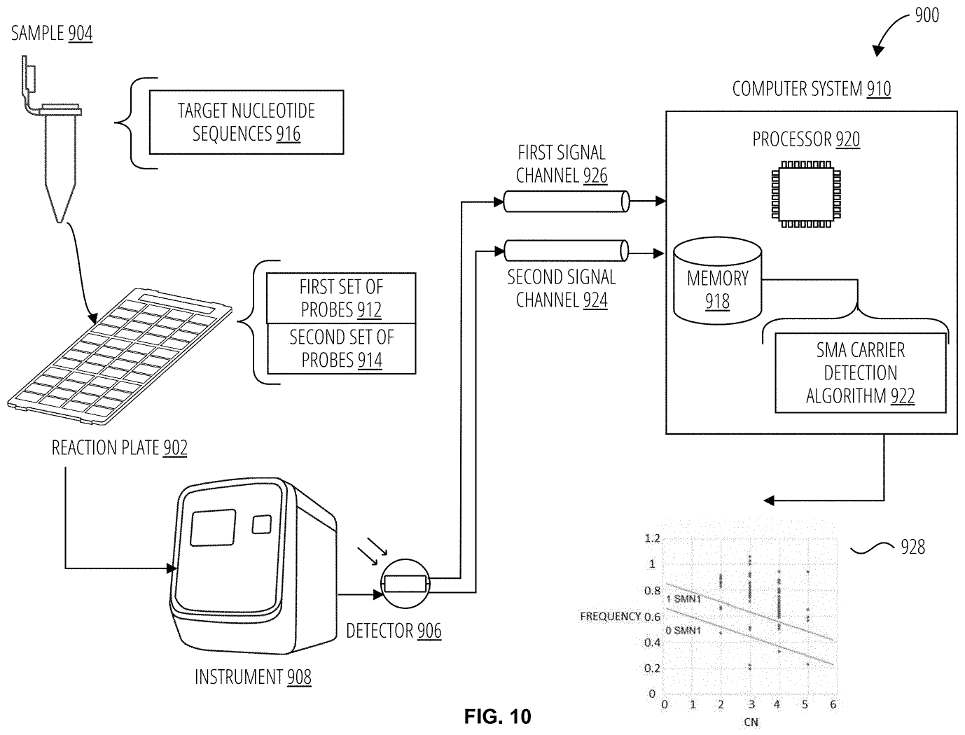

Referencing FIG. 10, a system 900 illustrates a system implementing the SMA carrier detection algorithm in accordance with one embodiment. In the system 900, a sample 904 comprising the target nucleotide sequences 916, polymerase, primers, and nucleotides are loaded on to a reaction plate 902. The reaction plate comprises are plurality of arrays running parallel reactions. A first set of probes 912 and a second set of probes 914 are present in each array and are utilized in the detection of the target nucleotide sequences 916. The first set of probes 912 targets a sequence that is different in the first and second target polynucleotide sequences. The second set of probes 914 targets a sequence that is identical in the first and second target polynucleotides sequences. The reaction plate 902 with the sample is then loaded into an instrument 908 to undergo several cycles of replication that includes a high heat phase (94-98° C. (201-208° F.)) that denatures the DNA strand breaking the hydrogen bonds between complementary bases, yielding two single-stranded DNA molecules. The denaturing phase is followed by the annealing phase where the reaction temperature is lowered to 50-65° C. (122-149° F.) for 20-40 seconds. The annealing phase allows for the annealing the probe sets to the target sequences in the DNA. The annealing phase is followed by labelling, e.g., via incorporation of one or more labelled nucleotides. The information for each probe is detected and reported as first data or second data. In some configurations, the instrument 908 may be operated in a set up where the first data is reported through a first signal channel 926 and the second data is reported through a second signal channel 924. The first data and the second data are reported to a computer system 910 comprising a processor 920 and memory 918, with the memory 918 comprising the instructions corresponding to the SMA carrier detection algorithm 922. Through the operation of the SMA carrier detection algorithm 922, the system 900 is able to generate a genotype map 928 indicating frequencies of a first target nucleotide sequence and a second target nucleotide sequence relative to the total predicted copy number of both target nucleotide sequences. The SMA carrier detection algorithm 922 adjusts the data based on the affinity of the each of the probes to the target nucleotide sequence. When the data is plotted, delineations can be made between groups of clusters indicating hyperplane regions based on the frequency of the two target sequences and the predicted total copy number of the two. These hyperplane regions indicate specific SMN1 genotypes corresponding to carrier and non-carriers.

In some embodiments, the first set of probes 912 may target different sequence such that the first set or probes indicates the presence of an SMN1 gene and an SMN2 gene. In some embodiments, the individual probes target the point mutation at exon 7 that differentiates a functioning copy of SMN1 with the copy of SMN2.

In some embodiments, the SMA carrier detection algorithm utilizes data collected through a multiplexed PCR reaction. In some embodiments, SMN1 gene sequence or part thereof is amplified in a multiplexed PCR reaction. In some embodiments, SMN2 gene sequence or part thereof is amplified in a multiplexed PCR reaction. In some embodiments, both of SMN1 and SMN2 gene sequences or part thereof are amplified in a multiplexed PCR reaction.

There can be benefits for PCR multiplexing, three of these benefits include increased throughput (more samples potentially assayed per plate), reduced sample usage, and reduced reagent usage (are dependent on the number of targets in the experiment). For example, if a quantitative experiment consists of only one target assay, running the target assay as a duplex with the normalizer assay, such as an endogenous control assay, will increase throughput, reduce sample required, and reduce reagent usage by half. If a quantitative experiment consists of two target assays, it may be possible to combine two target assays and the normalizer assay in a triplex reaction. In that case, the throughput increase, sample reduction, and reagent reduction will be even greater.

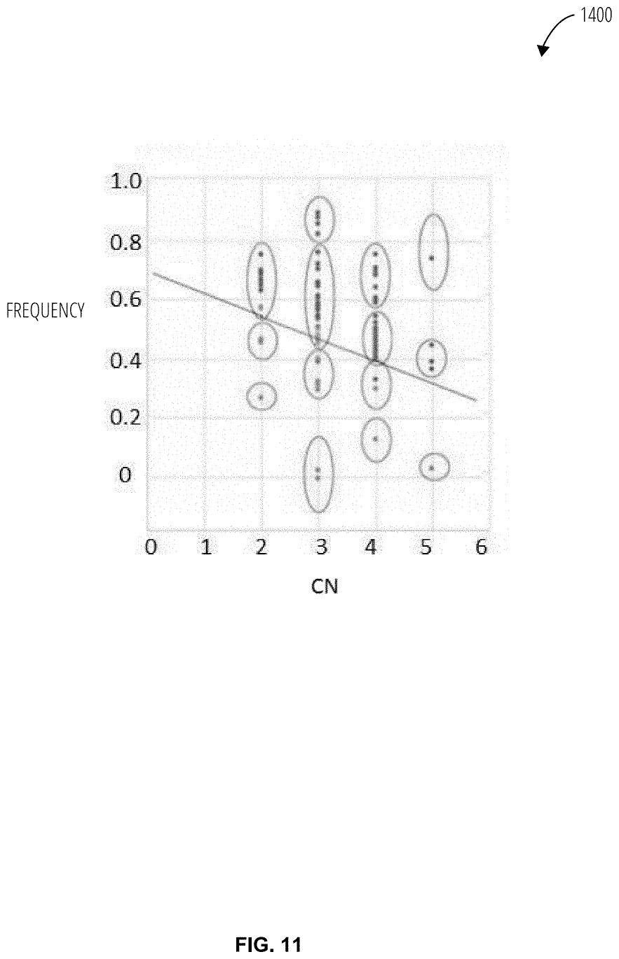

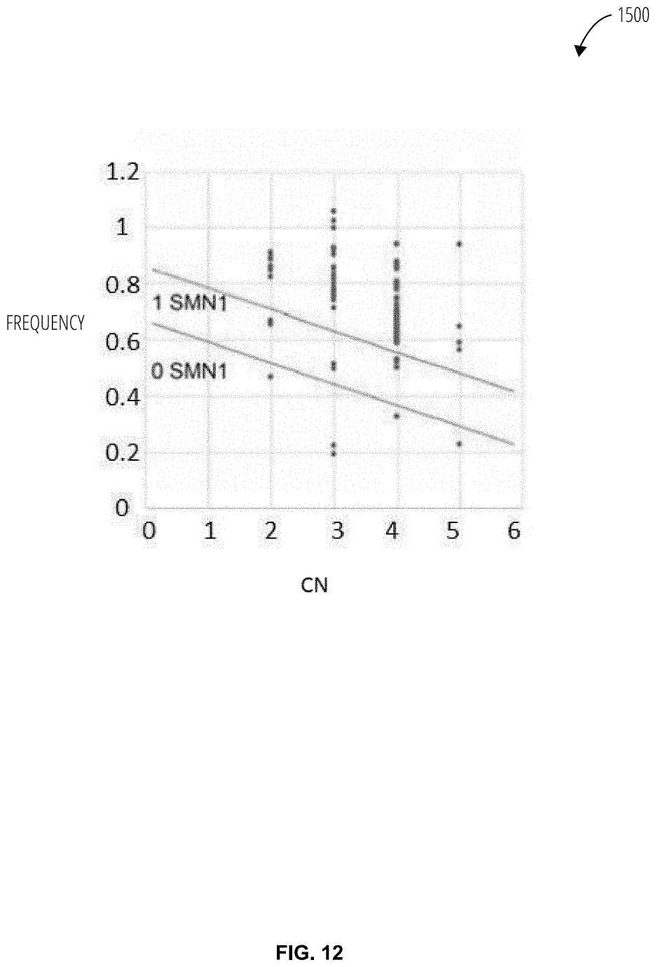

Referencing FIG. 11, a plot 1400 shows the initial distribution of reported data for the target sequences relative to the predicted copy number for the probe sets. A delineation is shown indicating the frequencies of SMN1 vs SMN2 where SMN2 are above the delineation while SMN1 is below the delineation. Although the results are show a distinction between the genes, there are some overlapping sections that could indicate potential carrier variations. In FIGS. 11 and 12, y-axis represents allele frequency of SMN1/SMN2 and x-axis represents combined SMN1 and SMN2 copy number.

Referencing FIG. 12, plot 1500 illustrates clear delineations in the reported data following the implementation of the SMA carrier detection algorithm of one embodiment. The adjusted data may allow delineations to be made indicating different carrier genotypes of SMA. The top delineation indicates a low value of SMN1 relative to the ratio of SMN1 and SMN2 indicating based on the predicted copy number that the top region corresponds to just copies of SMN2. The middle delineated region indicates the presence of just one copy of SMN1 possibly corresponding to a ‘1+1*’ or a ‘1+0’ carrier genotype.

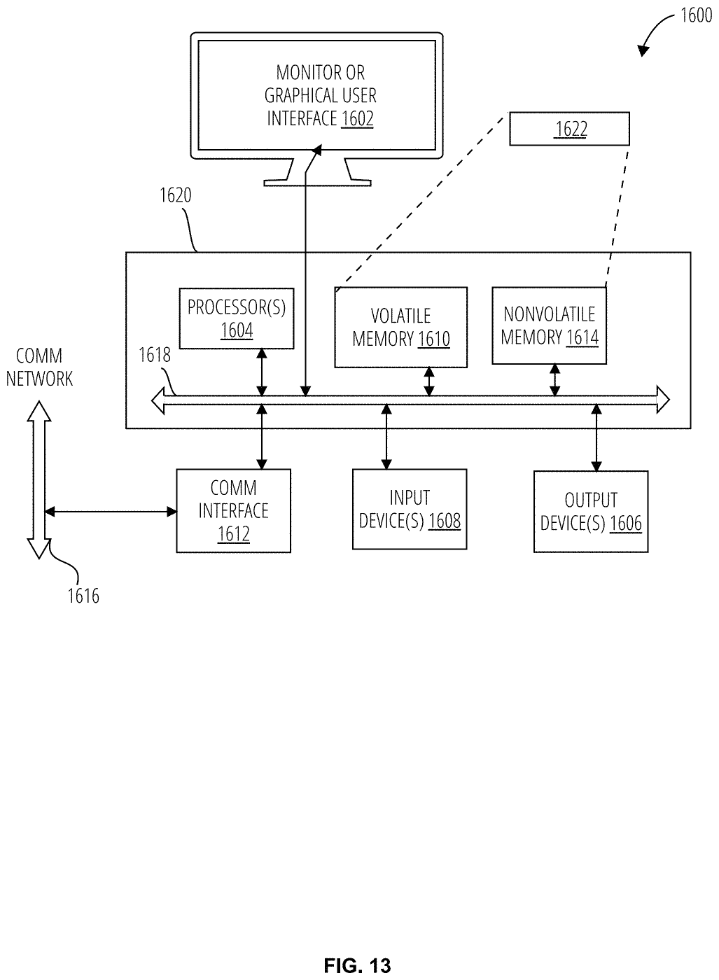

FIG. 13 is an example block diagram of a computing device 1600 that may incorporate some embodiments of the present disclosure. FIG. 13 is merely illustrative of a machine system to carry out aspects of the technical processes described herein, and does not limit the scope of the claims. One of ordinary skill in the art would recognize other variations, modifications, and alternatives. In one embodiment, the computing device 1600 typically includes a monitor or graphical user interface 1602, a data processing system 1620, a communication network interface 1612, input device(s) 1608, output device(s) 1606, and the like.

As depicted in FIG. 13, the data processing system 1620 may include one or more processor(s) 1604 that communicate with a number of peripheral devices via a bus subsystem 1618. In some embodiments, these peripheral devices includes input device(s) 1608, output device(s) 1606, communication network interface 1612, and a storage subsystem, such as a volatile memory 1610 and a nonvolatile memory 1614.

In some embodiments, the volatile memory 1610 and/or the nonvolatile memory 1614 stores computer-executable instructions and thus forming logic 1622 that when applied to and executed by the processor(s) 1604 implement embodiments of the processes disclosed herein.

In some embodiments, the input device(s) 1608 include devices and mechanisms for inputting information to the data processing system 1620. These may include a keyboard, a keypad, a touch screen incorporated into the monitor or graphical user interface 1602, audio input devices such as voice recognition systems, microphones, and other types of input devices. In various embodiments, the input device(s) 1608 may be embodied as a computer mouse, a trackball, a track pad, a joystick, wireless remote, drawing tablet, voice command system, eye tracking system, and the like. The input device(s) 1608 typically allow a user to select objects, icons, control areas, text and the like that appear on the monitor or graphical user interface 1602 via a command such as a click of a button or the like.

In some embodiments, the output device(s) 1606 include devices and mechanisms for outputting information from the data processing system 1620. These may include the monitor or graphical user interface 1602, speakers, printers, infrared LEDs, and so on as well understood in the art.

In some embodiments, the communication network interface 1612 provides an interface to communication networks (e.g., communication network 1616) and devices external to the data processing system 1620. The communication network interface 1612 may serve as an interface for receiving data from and transmitting data to other systems. Embodiments of the communication network interface 1612 may include an Ethernet interface, a modem (telephone, satellite, cable, ISDN), (asynchronous) digital subscriber line (DSL), FireWire, USB, a wireless communication interface such as BlueTooth or WiFi, a near field communication wireless interface, a cellular interface, and the like.

In some embodiments, the communication network interface 1612 is coupled to the communication network 1616 via an antenna, a cable, or the like. In some embodiments, the communication network interface 1612 may be physically integrated on a circuit board of the data processing system 1620, or in some cases may be implemented in software or firmware, such as “soft modems”, or the like.

In some embodiments, the computing device 1600 includes logic that enables communications over a network using protocols such as HTTP, TCP/IP, RTP/RTSP, IPX, UDP and the like.

The volatile memory 1610 and the nonvolatile memory 1614 are examples of tangible media configured to store computer readable data and instructions to implement various embodiments of the processes described herein. Other types of tangible media include removable memory (e.g., pluggable USB memory devices, mobile device SIM cards), optical storage media such as CD-ROMS, DVDs, semiconductor memories such as flash memories, non-transitory read-only-memories (ROMS), battery-backed volatile memories, networked storage devices, and the like. In some embodiments, the volatile memory 1610 and the nonvolatile memory 1614 are configured to store the basic programming and data constructs that provide the functionality of the disclosed processes and other embodiments thereof that fall within the scope of the present disclosure.

Logic 1622 that implements embodiments of the present disclosure may be stored in the volatile memory 1610 and/or the nonvolatile memory 1614. The logic 1622 may be read from the volatile memory 1610 and/or nonvolatile memory 1614 and executed by the processor(s) 1604. The volatile memory 1610 and the nonvolatile memory 1614 may also provide a repository for storing data used by the logic 1622.

In some embodiments, the volatile memory 1610 and the nonvolatile memory 1614 include a number of memories including a main random access memory (RAM) for storage of instructions and data during program execution and a read only memory (ROM) in which read-only non-transitory instructions are stored. In some embodiments, the volatile memory 1610 and the nonvolatile memory 1614 include a file storage subsystem providing persistent (non-volatile) storage for program and data files. In some embodiments, the volatile memory 1610 and the nonvolatile memory 1614 include removable storage systems, such as removable flash memory.

In some embodiments, the bus subsystem 1618 provides a mechanism for enabling the various components and subsystems of data processing system 1620 communicate with each other as intended. Although the communication network interface 1612 is depicted schematically as a single bus, some embodiments of the bus subsystem 1618 may utilize multiple distinct busses.

It will be readily apparent to one of ordinary skill in the art that the computing device 1600 may be a device such as a smartphone, a desktop computer, a laptop computer, a rack-mounted computer system, a computer server, or a tablet computer device. As commonly known in the art, the computing device 1600 may be implemented as a collection of multiple networked computing devices. Further, the computing device 1600 will typically include operating system logic (not illustrated) the types and nature of which are well known in the art.

### Kit

In some embodiments, the disclosure herein provides a kit for genotyping nucleic acids of a sample. The kit may include an array that has a first set of probes and a second set of probes that hybridize to a plurality of target polynucleotides. In some embodiments, the plurality of target polynucleotides include two or more different target polynucleotides, e.g., a first target polynucleotide and a second target polynucleotide. The first set of probes may hybridize to a first region that has a sequence different in the first and second target polynucleotides and the second set of probes hybridize to a second region that is identical in the first and second target polynucleotides. The first and second target polynucleotides may have sequence identity of at least 50%.

In some embodiments, the first region that is interrogated (or analyzed) by the kit provided herein has one or more base(s) that are different (variable) in the target polynucleotides and a sequence that is near or surrounding the variable base(s). In some embodiments, the first set of probes hybridizes to a sequence that is immediately 5′ or 3′ of the variable base(s). In some embodiments, the first set of probes terminates at a base immediately adjacent to the variable base(s). In some embodiments, the first set of probes comprises a sequence that is complementary to the variable base(s).

In some embodiments, the target polynucleotides that are interrogated by the kit herein are from different genes. In some embodiments, the target polynucleotides are not allelic variants of a gene. In some embodiments, the kit can be used to interrogate at least two genes, e.g., a clinically relevant gene and its related gene (e.g., a pseudogene). In some embodiments, the target polynucleotides that are interrogated by the kit herein have sequence identity of at least about 50%, about 55%, about 60%, about 65%, about 70%, about 75%, about 80%, about 85%, about 90%, about 95%, or about 99%.

In some embodiments, the kit provided herein further include instructions regarding the data collection and analysis thereof. In some embodiments, the instructions are in a computer-readable medium or in a computer. In some embodiments, the instructions contain a code for receiving data indicative of hybridization of the first and second sets of probes to the nucleic acids of a sample or application products thereof. In some embodiments, the instructions also include a code for determining a combined copy number of the target polynucleotides, e.g., the total copy number of the first and second polynucleotides in the nucleic acids of the sample. In some embodiments, the instructions include a code for determining a ratio of amounts of the target polynucleotides, e.g., the relative amount of the first and/or second polynucleotides from the nucleic acids of the sample. In some embodiments, the ratio refers to the relative amounts of the two target polynucleotides such as 1:1, 3:0, or 1:2. In some other embodiments, the ratio refers to an amount of one target polynucleotide relative to the total amount of target polynucleotides. Therefore, in one example, the relative amount of the first target polynucleotide can be determined by dividing the signals from the first target nucleotide by the sum of the signals from the first and second target nucleotides. Using the same way except the signal from the second target polynucleotide is divided by the sum of the signals, the relative amount of the second target polynucleotide can be determined. In some embodiments, the relative amount of one target polynucleotide (e.g., a clinically relevant gene) is used and sufficient for a carrier screening. In some other embodiments, the relative amount of two or more target polynucleotides (e.g., a clinically relevant gene and its pseudogene) are used for a carrier screening. In some embodiments, the instructions also include a code for determining a genotype of the target polynucleotides, e.g., the genotype of the first and/or second target polynucleotides from the nucleic acids of the sample.

In some embodiments, the disclosure herein provides a method of manufacturing an array for genotyping nucleic acids having a plurality of target polynucleotides. In some embodiments, the plurality of target polynucleotides include two or more different target polynucleotides, e.g., a first target polynucleotide and a second target polynucleotide. The first and second polynucleotides may have at least 50% of sequence identity. The manufacturing method may include providing a first set of probes to a substrate. The first set of probes may hybridize to a first region that comprises a sequence different in the target polynucleotides. The method may also include providing a second set of probes to the substrate. The second set of probes may hybridize to a second region that is identical in the target polynucleotides. In some embodiments, the first and second sets of probes are synthesized on a substrate. In alternative embodiments, the first and second sets of probes are attached to the substrate after being synthesized. In some embodiments, the first region has one or more base positions variable in the target polynucleotides and a sequence surrounding the variable base(s). In some embodiments, the first set of probes hybridizes to a sequence that is immediately 5′ of the variable base(s). In some embodiments, the first set of probes terminates at a base immediately adjacent to the variable base(s). In some embodiments, the first set of probes has a sequence that is complementary to the variable base(s).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

Screening for Spinal Muscular Atrophy Carriers

Spinal Muscular Atrophy (SMA) is a rare but devastating disease with autosomal recessive inheritance. In some populations, 1 in 50 people carry a mutation in SMN1 gene encoding a defective Survival Motor Neuron (SMN) protein. Carrier screening needs to accurately determine the number of functional SMN1 genes in an individual. Carrier detection is complicated by the presence of the highly homologous, but largely non-functional SMN2 gene. Of the 28,081 bp of the SMN1 and SMN2 genes, only 27 positions differ (21 single nucleotide substitutions and 6 small indels) accounting for only 38 nucleotides that differ between the gene sequences of SMN1 and SMN2.

In the examples provided herein, an array-based assay for genotyping and screening SMA carriers according to some embodiments was designed and performed. In particular, the array used herein had probe sets that were designed to discriminate the SMN1 and SMN2 genes based on these sequence differences. In addition, the array further contained 1,181 probe sets covering the SMN1 and SMN2 genes for determination of the combined gene copy number. The data showed that these probe designs can detect the relative numbers of the SMN1 and SMN2 genes as well as the overall copy number. The combination of these data were used for a novel algorithm to identify individuals carrying an SMA mutation, providing highly accurate and improved screening results as compared to any other methods available in the field.

### Example 1—Design of Probe Sets

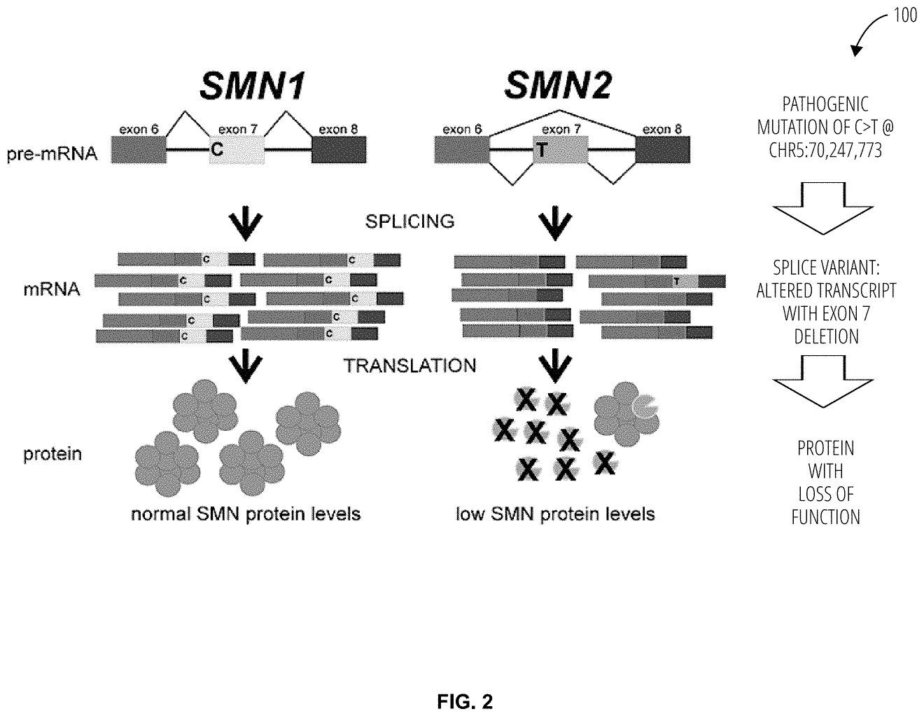

Comparison of the SMN1 and SMN2 genomic DNA sequences identified 27 positions with sequence differences between the two genes. These differences were used to design gene-specific probe sets. These positions are mostly intronic, but one lies within exon 7 and one lies within exon 8. (See FIG. 5) The exon 7 location is both a sequence difference between SMN1 and SMN2 and a site for a mutation that converts SMN1 into a nonfunctioning SMN2 gene. This mutation interferes with an exon-splice-junction and results in a transcript without exon 7. The most common type of carrier is an exon 7 deletion mutation, but the gene conversion mutation also occurs. FIG. 2 shows the four genomic positions with the probe sets that were used to detect the relative number of copies of SMN1 and SMN2.

Carrier determination needs an accurate assessment of total copy number of SMN1 and SMN2. The 1,181 copy number probe sets hybridize equally to the wild-type of two genes (SMN1 and SMN2), so the presumed baseline number of copies is 4 (two of each gene). Since the most-common deletion is exon 7, the design focused on the 35 copy number probe sets in and around exon 7.

### Example 2—Sample Preparation

### Example 2.1—While Genome Amplification and Target Amplification

Nucleic acid samples for the genotyping and carrier screening for SMA were prepared and processed generally by following the protocols available for CarrierScan™ Assay Kit from Thermo Fisher Scientific (catalog number: 931931) and GeneTitan instrument (id.). Briefly, a biological sample (e.g., whole blood, saliva or cells) obtained from an individual and genomic DNA (gDNA) was isolated from the biological sample. The isolated gDNA, which was diluted to 5 μg/μL, was used to amplify the gDNA and also perform multiple PCR that is to amplify target polynucleotides. As for the amplification of gDNA sample, 20 μL of the diluted gDNA and 20 μL of control DNA were separately aliquoted to a plate (e.g., Applied Bioscience Gene 96 square well plate). After sealing and spinning the plate, the PCR reaction was performed with the reagents as indicated in the manufacture's protocol. As for the target amplification, 10 μL of diluted gDNA and 10 μL of reference DNA were aliquoted to a separate 96-well plate. This plate was sealed, spun and proceeded to a PCR reaction as instructed in the protocol. In the mPCR reaction, sequence specific primers were used to amplify SMN1 and/or SMN2 genes or parts thereof. The target amplification was performed to amplify the regions of SMN1 and SMN2 genes that were targeted by the probes. Thus, in one example, certain regions from both genes that were targeted by a first set of probes, which were used to determine relative amounts of both genes, were amplified. In addition, the regions that were targeted by a second set of probes, which were used to measure a combined copy of both genes, were also amplified. In this example, the regions covering exon 7 and/or exon 8 of SMN1 and SMN2 were amplified via the mPCR reaction. The amplified DNA samples and mPCR reaction plate were stored at −20° C. when needed.

### Example 2.2—Fragmentation of Amplified DNAs

After the whole genome amplification and mPCR reaction were completed, 10 μL of the mPCR reaction products from each well of the 96 plate was carefully transferred into the corresponding well of the whole genome amplification plate. The samples were well mixed by pipetting up and down and pulse spun down. A master mix for fragmentation, which contained Axiom Frag Enzyme (Thermo Fisher Scientific), was aliquoted to each well of the mixed DNA samples. The samples were incubated at 37° C. for 45 minutes for fragmentation reaction. Once the fragmentation reaction was over, the stop solution according to the manufacture's protocol was added to the sample plate to stop the fragmentation reaction. After the fragmentation reaction was completed, a master mix for precipitating the sample DNAs was added in individual wells of the plate, which was followed by addition of 2-propanol to each well. The precipitated DNA pellets in each well were dried and stored until the next step.

### Example 3—Denaturation of Fragmented DNAs

A resuspension buffer was added to each well of the sample plate that contained precipitated DNAs. A hybridization master mix was later added to each well having suspended DNAs, following the manufacturer's protocol. The sample plate was then proceed to a denaturation step (10 min, 95° C. and 3 min, 48° C.) using a thermal cycler as recommended by the manufacturer.

### Example 4—Hybridization and Staining

The steps of hybridization, staining and ligation were performed using the GeneTitan MC instrument (Thermo Fisher Scientific) and protocols provided by the manufacturer. Following the protocols, the master mixes for staining, ligation and stabilization were prepared in advance. In this example presented herein, the staining master mixes had two separate solutions as the assay employed 2-channelle system, utilizing two labelling molecules for staining.

The plate having denatured DNAs was loaded into the GeneTitan MC instrument along with the hybridization array having probes. The automated process by the instrument transferred the denatured DNAs to the hybridization array plate and incubated the array plate a controlled condition for hybridization. After the hybridization, the array plate was washed a few times with the wash buffer and underwent two separate steps of staining (Stain 1 and Stain 2) as part of the automated process. After the hybridization and washing, the master mix for the first staining step (Stain 1) was added to the array plate, which was followed by the addition of the ligation master mix. The first staining master mix had A/T-labelled with a first label and the first label would be added to the probe if the template had A or T. The second staining mater mix for Stain 2 had G/C-labeling with a second label and the labelled G or C would be added to the probe if the template had G or C. This template specific ligation would label the probes that hybridized to its corresponding target polynucleotides.

### Example 5—Scanning

Once the array plate went through the fluidics stage of the foregoing process, it was moved to the instrument's imaging station and scanned for data collection.

A number of controls including a reference genomic DNA were used to access the quality of each reaction step as well as sample quality.

### Example 6—Algorithm

Provided herein is a description of one implementation of the SMN detection algorithm operating on a computer system as a program. In this particular example, SMN1 and SMN2 were detected via a two-channel system (channel a and b) as indicated below (see the last two columns of the following txt input file). The frequency measured and calculated for a target sequence measured in each channel is indicated as an allele frequency in this example. For instance, a frequency measured from channel B is shown as B allele frequency (BAF) below. However, it should be noted that this frequency is for a different gene measured from each channel, not for a different allele. Therefore, the allele frequency provided in the disclosure herein, e.g. B allele frequency (BAF) should be be considered as a pseudo BAF, which is indicative of a frequency of one of related genes, not an allelic variant of a single gene.

Example CarrierScan.SMN.v1.AB_probesets.txt input file:

# % array_type = CarrierScan
# % factor1 = centering factor
# % factor2 = negative scaling factor
# % factor3 = positive scaling factor
# % factor2_threshold =<0.485
# % factor3_threshold =>0.515

| | | | factor2 | factor3 (to | | | |
|---|---|---|---|---|---|---|---|
| | | | (to scale | scale | | | |
| | | factor1 | when | when | | | |
| | | (median | B/(A + | B/(A + | | smn1_ | smn2_ |
| probeset_id | affy_snp_id | centering) | B) <0.485 | B) >0.515 | cn_region | channel | channel |
| AX- | Affx- | 0.007 | 2.7 | 2.4 | SMN1_uc01 | A | B |
| 123335186 | 26906853 | | | | 1crr.2_7 | | |
| AX- | Affx- | −0.055 | 2.2 | 2 | SMN1_uc01 | A | B |
| 123339936 | 26906853 | | | | 1crr.2_7 | | |
| AX- | Affx- | −0.066 | 4.2 | 3 | SMN1_uc01 | B | A |
| 169310928 | 122979826 | | | | 1crr.2_7 | | |
| AX- | Affx- | −0.054 | 3.4 | 3 | SMN1_uc01 | B | A |
| 169324443 | 122979826 | | | | 1crr.2_7 | | |
| AX- | Affx- | −0.007 | 1.6 | 1.2 | SMN1_uc01 | A | B |
| 158628589 | 206872225 | | | | 1crr.2_7 | | |
| AX- | Affx- | 0.043 | 4.4 | 2.4 | SMN1_uc01 | A | B |
| 169257715 | 206872225 | | | | 1crr.2_7 | | |

When SMN1 is listed as the A channel, the method would end up computing 1−BAF=A/(A+B), but in this document it is described as computing BAF and then in the very end (after the probe sets for each given marker are computed) it gets complemented.

Channel A is the signal obtained from the genotyping summary file for channel A for that probe set.

Another item in the pseudo code below is that we want final BAFs to be between 0 and 1. Due to the scaling they can go below 0 or above one, in which case we simply reset them.

The procedure is shown here for six probe sets, corresponding to 3 markers (affy_snp_id)
1. Raw “B Allele Frequency” (rBAF) calculation
   a. Read in all probe sets from probeset_id column in *AB_probesets.txt
   b. Find the intensityA and intensityB value from AxiomGT1.summary.a5 (hdf5 format)
      i. RowNames table
         1. intensityA=<probeset_id>-A
         2. intensityB=<probeset_id>-B
      ii. ColNames table
         1. Index of each row will give the cel_file name in order left-right shown in the data table
   c. For each sample calculate the rBAF using the Data table:

$$rBAF = raw BAF = \frac{( intensityB )}{( intensityA ) + ( intensityB )}$$

2. Center raw BAF
   a. Find the associated centering factor from factor1 column in *AB_probesets.txt
   b. For each sample:probeset rBAF center by:

cBAF=centered raw BAF=0.5+(rBAF−factor1)

In some embodiments, factor 1 can be a base line Bi. In some embodiments, rBAF is computed for samples that have 2 copies of SMN1 and 2 copies of SMN2, and therefore can be considered as having 2 A alleles and 2 B alleles. In such embodiments, factor 1 is the median rBAF across these samples. As a result the median BAF across these samples is 0.5.
3. Scale centered BAF
   a. If cBAF is <0.485, then find factor2 column in *AB_probesets.txt
      i. For each sample:probeset cBAF scale by:
   scBAF=scaled centered raw BAF=0.485−(0.485−cBAF)×factor2
   b. If cBAF is >0.515, then find factor3 column in *AB_probesets.txt
      i. For each sample:probeset cBAF scale by:
   scBAF=scaled centered raw BAF=(cBAF−0.515)×factor3+0.515
   c. Else (0.515≥cBAF≥0.485)
      scBAF=scaled centered raw BAF=cBAF
   d. Scale scBAF to between 0 and 1:
      i. If scBAF>1, please set scBAF to 1,
      ii. If scBAF<0, please set scBAF to 0,
      iii. Else, use the calculated scBAF in the following steps.

Here the algorithm started grouping together probe sets that measure the exact same marker. Affy_snp_if is an ID that references a given marker. So after calculating all the scBAFs for a given marker, take the median of that measurement for each marker.
4. Median scBAF for affy_snp_id
   a. For each probeset_id, find the associated affy_snp_id in *AB_probesets.txt
   b. Calculate the median for the affy_snp_id for each sample=mBAF
   In this step below the medians get complemented if they go in the opposite direction.
5. Check channel to determine “real BAF” direction, by checking the smn1_channel column for each affy_snp_id:
   a. If smn1_channel=A:
      i. mBAF_<affy_SNP_ID>=1−mBAF
   b. if smn1_channel=B:
      i. mBAF_<affy_SNP_ID>=mBAF
      ii.

Here multiple markers were looked and the median of medians across multiple markers in the region was determined. To make the final call 3 measurements were used.
6. Median mBAF for cn_region
   a. For each cn_region, find associated affy_snp_id in *AB_probesets.txt
   b. Calculate the median for each cn_region based by using the median BAF for each
   <affy_snp_id>per sample=mBAF_<cn_region>
   Median (mBAF_<affy_SNP_ID1>, mBAF_<affy_SNP_ID2>, . . . )

In step 7 the value for each affy_snp_is (marker is computed as well as the median across markers). —called below mBAF for the cn_region.
7. Reports
   a. <analysis_name>.SMN_ABreport.txt (example:mPCR90.SMN_ABreport.txt)
      i. cel_files =name of cel files
      ii. mAB_<affy_snp_id>=mBAF for the affy_snp_id
      iii. mAB_<cn_region>=median mBAF for the cn_region
         Example Analysis report mPCR90.SMN_ABreport.txt

| #%array_type=CarrierScan | | | | |
|---|---|---|---|---|
| #%batch_name=mPCR90 | | | | |
| #%library_version=CarrierScan_SMN.v1 | | | | mAB_SM |
| #%other_headers . . . | mAB_Affx- | mAB_Affx- | mAB_Affx- | N1_uc011c |
| cel_files | 26906853 | 122979826 | 206872225 | rr.2_7 |
| NA03252_mPCR90_CSTrainingSamples_C | 0.500 | 0.507 | 0.493 | 0.500 |
| arrierScan96_20171025_F05.CEL | | | | |
| NA03461_mPCR90_CSTrainingSamples_C | 0.363 | 0.385 | 0.560 | 0.385 |
| arrierScan96_20171025_H04.CEL | | | | |
| NA03770_mPCR90_CSTrainingSamples_C | 0.384 | 0.361 | 0.552 | 0.384 |
| arrierScan96_20171025_A05.CEL | | | | |
| NA03815_mPCR90_CSTrainingSamples_C | 0.494 | 0.501 | 0.495 | 0.495 |
| arrierScan96_20171025_G05.CEL | | | | |

### Example 8—Report the Calls

The copy number (CN) state (of SMN1+SMN2) is computed for the region. Each copy number sate has a different threshold at which “SMN1 has less than 2 copies” is called, as shown in the table below: Table 1: threholds.

TABLE 1

| Copy | | | | |
|---|---|---|---|---|
| Number | | | | |
| (CN) | 2 | 3 | 4 | 5 or more |
| Threshold | 0.55 | 0.44 | 0.36 | 0.3 |
| for calling 1 | | | | |
| copy of | | | | |
| SMN1 | | | | |

The additional table (below) shows the expected BAF for each CN and state of SMN1. Table 2: expected vales for each CN state.

TABLE 2

| | TOTAL | CN | | |
|---|---|---|---|---|
| SMN1 CN | 2 | 3 | 4 | 5 |
| 0 | 0 | 0 | 0 | 0 |
| 1 | 0.5 | 0.33 | 0.25 | 0.2 |
| 2 | 1 | 0.66 | 0.5 | 0.4 |
| 3 | na | 1 | 0.75 | 0.6 |
| 4 | na | Na | 1 | 0.8 |
| 5 | na | Na | Na | 1 |

Note that the thresholds in Table 1 were empirically derived and while they were driven by the theoretical values in Table 2—there is no formula that calculates the actual thresholds used from the theoretical values.

The threshold are applied as follows and four possible results are reported.
1) When the median mBAF for the cn_region is less or equal than the value listed in the above table for the respective copy number the sample is designated a “carrier,” e.g. CN of SMN1 is 1 or less.

Else
2) When the BAF for Affx-206872225 is less than the thresholds in the above table a “conversion event is called”.

This is interpreted as a “conversion” event. The conversion event is reported—and the sample is also a carrier SMN1 is present but the critical allele for the above marker mutated to the value that SMN2 has—making the gene inactive.

Else
3) There is one marker inside exon 8—when the BAF for only that marker is less than the corresponding threshold in the above table “an exon 8 deletion” is called. We are not sure if customers will interpret this as a carrier, but they requested this to be reported.

Else
4) Nothing gets reported.

### Parameters/Options:

| Input(s) | Output(s) |
|---|---|
| --smn-ab-probe sets | <batch_name>.SMN_ABreport.txt |
| <CarrierScan.SMN.v1.AB_probe- | |
| sets.txt> | |
| --summary-a5-file | |
| <AxiomGT1.summary.a5> | |
| --out-dir | |
| (optional, best practices = | |
| <library_version>, | |
| default = “analysis_files” | |
| --set-analysis-name | |
| (optional, best practices = | |
| <batch_name>, | |
| default = “apt2-smn-ab” | |

### Example 9—Calling SMN1/SMN2 Copy Number

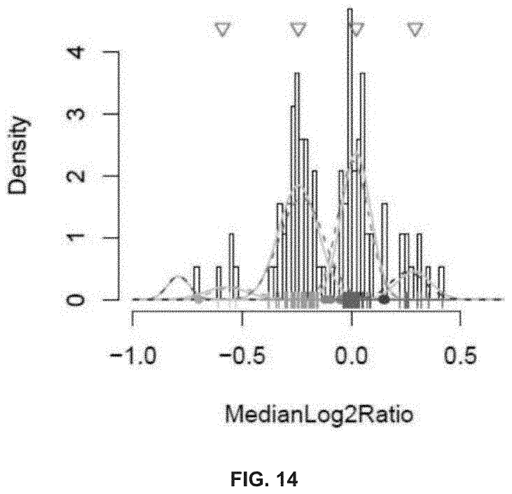

FIG. 14 shows the distribution of copy number for 96 representative samples. The peak at a log2 ratio of 0.0 represents the individuals with 4 copies of SMN1 and SMN2 combined. The CNVMix algorithm used in the examples herein identifies 4 copy number states in this set of samples, 2, 3, 4 and 5. Surprisingly, a large number of samples have 3 copies of these genes. Total copy number is important. For example, a sample that has equal amounts of SMN1 and SMN2 at a total copy number of 2 is clearly a carrier, but at a total copy number of 4, that same ratio implies a non-carrier.

### Example 10—Identification of Carriers for SMA

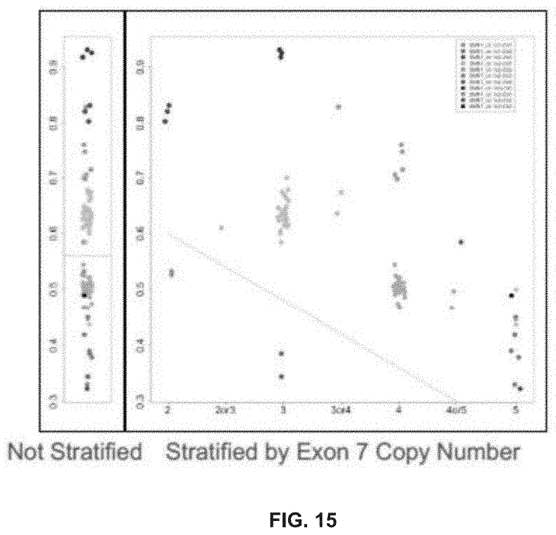

Frequency of SMN1 and SMN2 genes, which is marked as a BAF (B-allele frequency) in the examples presented herein is the metric that reports the relative amounts of SMN1 and SMN2. The left panel of FIG. 15 shows that BAF alone cannot separate carriers (red dots) from noncarriers. By stratifying the data by total copy number and BAF, there is a clear separation of carriers and non-carriers (dotted line), forming the basis for a carrier detection algorithm. Preliminary application of the SMA detection algorithm on a data set of 493 samples had no false negative calls. A moderate rate of false positive calls was made, but this is acceptable in a screen. The examples presented herein clearly prove that the assay and algorithm provided herein provide highly accurate and significantly improved screening results for carrier screening.

### Example 11—Display of Copy Number of SMA Genes

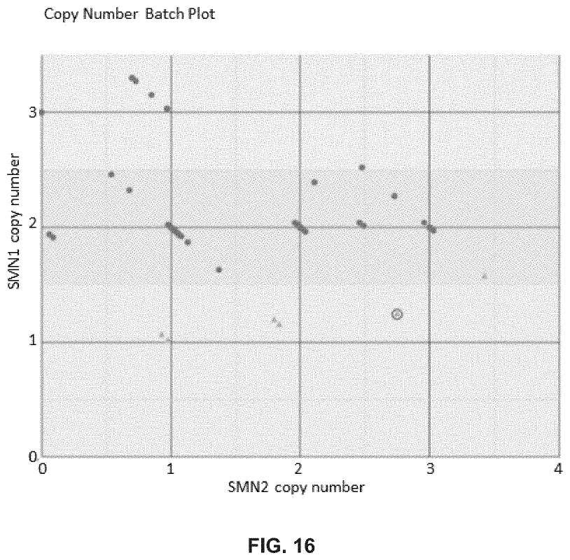

In some embodiments, the copy number calculated according to Example 10 can displayed, for example, in a plot such as shown in FIG. 16 displays (e.g., SMN1 copy number at y-axis and SMN2 copy number at x-axis). Based on the frequency of each gene, the copy number inferred from the frequency is plotted in a hyperplane format. In one example, the samples suspected to be SMN1 carrier are plotted at value 1.5 or below on the y-axis. Thus, samples marked as triangle in FIG. 16 are suspected to be a carrier. Each suspected carrier has a different copy number of SMN2 as shown in the x-axis. With this display in which the data is transformed to more readily understandable, user-friendly format or interface, one can easily determine a carrier status of a sample.

In some embodiments, the copy number of one or more of the target genes can be displayed in any user interface form on a screen (created locally or via remotely from a network) or in a print. Such a display can be in form of table or text.

1.-48. (canceled)

49. A computer-implemented method for genotyping a mixture of nucleic acids, said mixture comprising a first target polynucleotide and a second target polynucleotide having a sequence identity of at least 50% to the first target polynucleotide, the method comprising:
obtaining, by a computer comprising a processor, first data of an intensity measurement from a first set of probes, wherein the first set of probes targets a sequence that is different in the first and second target polynucleotide sequences;
obtaining, by the computer, second data of an intensity measurement from a second set of probes, wherein the second set of probes targets a sequence that is identical in the first and second target polynucleotides sequences;
determining, by the processor, from the first data a ratio of the first and second target polynucleotides in the mixture;
determining, by the processor, from the second data a combined copy number of the first and second target polynucleotides in the mixture; and
determining, by the processor, a genotype of at least one of the first and second target polynucleotides.

50. The method of claim 49, wherein the first and second sets of probes are provided in an array.

51. (canceled)

52. The method of claim 49, wherein said ratio of the first and second target polynucleotides is a ratio of the first and second target polynucleotides in a human genome.

53. The method of claim 49, wherein said combined copy number of the first and second target polynucleotides is a combined genomic copy number of the first and second target polynucleotides in a human genome.

54. The method of claim 49, wherein the first and second target polynucleotides are from different genes.

55. The method of claim 49, wherein the first and second target polynucleotides are not allelic variants of a gene.

56. The method of claim 49, wherein the target polynucleotides are Survival Motor Neuron 1 (SMN1) and Survival Motor Neuron 2 (SMN2) genes or part thereof.

57. The method of claim 56, wherein the first target polynucleotide is found in the SMN2 gene and a variant of SMN1 gene with a mutation in and around exon 7.

58. The method of claim 56, wherein the second target polynucleotide is found in the SMN1 gene.

59. The method of claim 8, wherein the first set of probes comprises at least four probe sets and each probe set corresponds to a sequence that is different in SMN1 and SMN2 genes.

60. The method of claim 59, wherein the at least four probe sets targeting variants in and around exon 7 of the SMN1 gene target the following regions: a region containing chromosome 5:70,247,773C>T site, a region containing chromosome 5: 70,247,921A>G site, a region containing chromosome 5: 70,248,036A>G site, and a region containing chromosome 5: 70,248,501G>A.

61. (canceled)

62. The method of claim 49 further comprising:
receiving data of signals from the array, wherein in the first set of probes the first target polynucleotide is reported;
calculating an average intensity value for the probe sets and determining a standard deviation between the average intensity values;
calculating a raw frequency of the target polynucleotides;
calculating a centered frequency of the target polynucleotides from the respective raw frequency;
calculating a scaled and centered frequency of the target polynucleotides from the respective centered frequency;
calculating a median frequency of the target polynucleotides from an affinity value of each probe set for the target polynucleotides and predicted copy number (CN);
delineating hyperplanes corresponding to presence of no copy of the target polynucleotides in the mixture, one copy of the target polynucleotides gene in the mixture, and two copies of the target polynucleotides in the mixture; and
correlating quantity of probe set clusters within the hyperplanes as a statistical indication of the number of copies of the target polynucleotides in the mixture.

63. The method of claim 62 further comprising:
displaying the number of copies of one or more of the target polynucleotides in the mixture.

64. The method of claim 62, wherein the method further comprises:
scaling the scaled and centered frequency by:
setting the scaled and centered frequency to 1 in response to the scaled and centered frequency being greater than 1; and
setting the scaled and centered frequency to 0 in response to the scaled and centered frequency being less than 0; and
determining the direction of the frequency by subtracting a median frequency for the first target polynucleotide, and using the median frequency value for the second target polynucleotide.

65. The method of claim 62, wherein calculating the raw frequency for the probe sets further comprises dividing an intensity for the second target polynucleotide by the sum of an intensity for the first target polynucleotide and an intensity for the second target polynucleotide.

66. The method of claim 62, wherein calculating the raw frequency for the probe sets further comprises dividing an intensity for the first target polynucleotide by the sum of an intensity for the first target polynucleotide and an intensity for the second target polynucleotide.

67. The method of claim 62, wherein calculating the centered frequency for the probe sets from the raw frequency further comprises subtracting the standard deviation from the raw frequency and then adding an ideal frequency ratio of 0.5, the ideal frequency being the frequency between the first and second target polynucleotides.

68. The method of claim 62, wherein calculating the scaled and centered frequency for the probe sets from the centered frequency further comprises:
multiplying the difference between the centered frequency and a first alpha cutoff value by a first scaling factor and then subtracting this value from the first alpha cutoff value, in response to the centered frequency being less than the first alpha cutoff value;
multiplying the difference between the centered frequency and a second alpha cutoff value by a second scaling factor and then adding this value to the second alpha cutoff value, in response to the centered frequency being greater than the second alpha cutoff value; and
identifying the centered frequency as the scaled and centered frequency in response to the centered frequency being equal or within a range formed by the first alpha cutoff value and the second alpha cutoff value.

69. The method of claim 62 further comprises:
plotting the scaled and centered frequency for the probe sets against their predicted copy number on a graph;
delineating the hyperplanes in the graph corresponding to presence of no copy of the target polynucleotides in the mixture, one copy of the target nucleoids in the mixture, and two copies of the target nucleotides in the mixture; and
correlating the quantity of probe set clusters within the hyperplanes as the statistical indication of the number of copies of the target nucleotides in the mixture.

70. The method of claim 62 further comprising:
normalizing the raw frequency for each of the probe sets.

71. The method of claim 70, wherein normalizing the raw frequency for the probe sets further comprises:
calculating a centered frequency for the probe sets from the raw frequency by subtracting the standard deviation from the raw frequency and then adding an ideal frequency ratio of 0.5, the ideal frequency being the raw frequency between the first and second target polynucleotides;
calculating a scaled and centered frequency for the probe sets from the centered frequency by:
multiplying the difference between the centered frequency and a first alpha cutoff value by a first scaling factor and then subtracting this value from the first alpha cutoff value, in response to the centered frequency being less than the first alpha cutoff value;
multiplying the difference between the centered frequency and a second alpha cutoff value by a second scaling factor and then adding this value to the second alpha cutoff value, in response to the centered frequency being greater than the second alpha cutoff value; and
identifying the centered frequency as the scaled and centered frequency in response to the centered frequency being equal or within a range formed by the first alpha cutoff value and the second alpha cutoff value.

72.-92. (canceled)
calculating a scaled and centered frequency of the target polynucleotides from the respective centered frequency, a first alpha cutoff value, a second alpha cutoff value, the first scaling factor, and the second scaling factor;
calculating a median frequency of the target polynucleotides from an affinity value of each probe set for the target polynucleotides and predicted copy number (CN);
delineating hyperplanes corresponding to presence of no copy of the target polynucleotides, one copy of the target polynucleotides, and two copies of the target polynucleotides;
correlating quantity of probe set clusters within the hyperplanes as a statistical indication of the number of copies of the target polynucleotides; and
displaying the number of copies of one or more of the target polynucleotides in the mixture.


SEQUENCE LISTING (3 sequences)

SEQ ID NO: 1 - DNA, 850 bp, Artificial Sequence
  Synthetic SMN1 base sequence 500
tgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatgtctatatagctattttttttaacttcctttattttccttacagggtttcagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttaaaaagttgaaaggttaatgtaaaacaatcaatattaaagaattttgatgccaaaactattagataaaaggttaatctacatccctactagaattctcatacttaactggttggttatgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaag

SEQ ID NO: 2 - DNA, 1650 bp, Homo sapiens
  misc_feature: SMN1
taaatctcagctcactgcagcctccgcctcctgggttcaagtgattctcctgcctcagcctcccaagtagctgggattagaggtccccaccaccatgcctggctaattttttgtactttcagtagaaatggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatgtctatatagctattttttttaacttcctttattttccttacagggtttcagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttaaaaagttgaaaggttaatgtaaaacaatcaatattaaagaattttgatgccaaaactattagataaaaggttaatctacatccctactagaattctcatacttaactggttggttatgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactggggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtata

SEQ ID NO: 3 - DNA, 1650 bp, Homo sapiens
  misc_feature: SMN2
taaatctcagctcactgcagcctccgcctcctgggttcaagtgattctcctgcctcaacctcccaagtagctgggattagaggtccccaccaccatgcctggctaattttttgtactttcagtagaaacggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatatctatatagctattttttttaacttcctttattttccttacagggttttagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttagaaagttgaaaggttaatgtaaaacaatcaatattaaagaattttgatgccaaaactattagataaaaggttaatctacatccctactagaattctcatacttaactggttggttgtgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtata